(19) United States
(12) Patent Application Publication
(10) Pub. No.: US 2024/0083949 A1
(43) Pub. Date: Mar. 14, 2024

(54) CELL PENETRATING PEPTIDE COMPOSITIONS AND METHODS THEREOF

(71) Applicant: The Regents of the University of California, Oakland, CA (US)

(72) Inventors: Min Xue, Chino Hills, CA (US); Siwen Wang, Jurupa Valley, CA (US); Nathan Guevara, Pomona, CA (US)

(21) Appl. No.: 18/233,684

(22) Filed: Aug. 14, 2023

Related U.S. Application Data
(60) Provisional application No. 63/398,438, filed on Aug. 16, 2022.

(51) Int. Cl. C07K 7/64; A61K 47/64

(52) U.S. Cl. CPC C07K 7/64; A61K 47/64

(57) ABSTRACT

Certain embodiments of the invention provide cell penetrating peptides. Certain embodiments of the invention provide methods and compositions for intracellular delivery. Certain embodiments of the invention provide methods and compositions for targeted delivery.

### CROSS REFERENCE TO RELATED APPLICATION

This application claims priority to U.S. Provisional Application No. 63/398,438 filed on 16 Aug. 2022. The entire content of the application referenced above is hereby incorporated by reference herein.

### GOVERNMENT FUNDING

This invention was made with government support under EB025393 and GM138214 awarded by the National Institutes of Health. The government has certain rights in the invention.

### BACKGROUND OF THE INVENTION

Over the past few decades, a very diverse panel of cell-penetrating peptides (CPPs) has been developed. Early examples such as the transactivator of transcription (TAT) peptide and RGD sequence have proven capable of delivering various cargo molecules, and they continue to be widely employed to date. More recently, advanced sequences, such as penetratin, iRGD, and CPP12, have demonstrated superior delivery efficacy. Currently, improving the cell-penetrating ability, intracellular targeting, and biocompatibility of these CPPs remains a very active and attractive research field. There is a need for developing new vehicles and modes for transmembrane intracellular delivery.

### SUMMARY OF THE INVENTION

Certain embodiments of the invention provide a macrocyclic compound comprising a pentapeptide segment X₁-X₂-X₃-X₄-X₅, wherein:
X₁, X₂, X₃, X₄, and X₅ are each independently a residue of Phe, Tyr, Thr, Ser, or homo-Ser;
wherein Phe or Tyr is optionally substituted with one or more halo or hydroxy group on the phenyl ring; and
wherein Thr, Ser, or homo-Ser is optionally substituted with one or more halo, hydroxy, (C₁-C₆)alkyl, (C₃-C₆)cycloalkyl, aryl, or heteroaryl, wherein the (C₁-C₆)alkyl, (C₃-C₆)cycloalkyl, aryl, or heteroaryl is optionally substituted with one or more halo or hydroxy group;
or a peptidyl residue or a salt thereof,

provided the pentapeptide segment is not Tyr-Tyr-Thr-Tyr-Thr (SEQ ID NO:1).

In certain embodiments, the macrocyclic compound is a macrocyclic compound, from N terminal to C terminal, having formula I:

(Rₙ)₂N-c[X₀-X₁-X₂-X₃-X₄-X₅-X₆]—C(═O)—R_{c} (I)

wherein:

X₀ is a residue of an amino acid,

X₆ is a residue of an amino acid,

each Rₙ is independently H, (C₁-C₆)alkyl, or (C₁-C₆)alkanoyl, and

R_{c} is hydroxy or —N(R_{f})₂, wherein each R_{f} is independently H or (C₁-C₆)alkyl,

or a peptidyl residue or a salt thereof.

In certain embodiments, the macrocyclic compound is a macrocyclic compound having formula II:

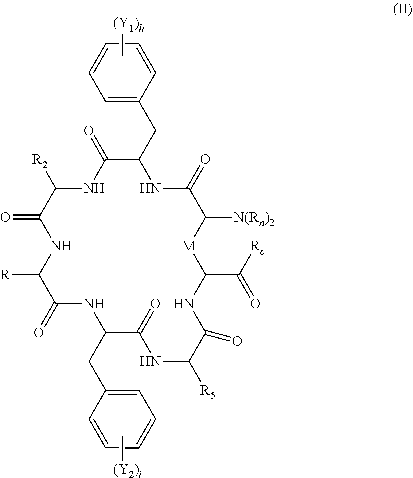

wherein
R₂ and R₅ are each independently (C₁-C₃)alkyl substituted with one or more hydroxy groups,
R is benzyl optionally substituted with one or more halo or hydroxy groups on the phenyl ring, or (C₁-C₃)alkyl substituted with one or more hydroxy groups,
each Y₁ is independently halo or hydroxy,
each Y₂ is independently halo or hydroxy,
h and i are each independently 0, 1, 2, 3, 4, or 5,
each Rₙ is independently H, (C₁-C₆)alkyl, or (C₁-C₆)alkanoyl,
R_{c} is hydroxy or —N(R_{f})₂,
each R_{f} is independently H or (C₁-C₆)alkyl,
M is divalent, branched or unbranched, saturated or unsaturated, hydrocarbon chain having from 2 to 16 carbon atoms, wherein one or more of the carbon atoms is optionally replaced by (—O—), (—S—), (—NRₐ—), (C₃-C₈)cycloalkyl, aryl, or heteroaryl, wherein the chain is optionally substituted on carbon with one or more substituents selected from the group consisting of halo, hydroxy, mercapto, oxo (═O), and thioxo(═S); wherein Rₐ is a H or (C₁-C₆)alkyl,
or a peptidyl residue or a salt thereof.

Certain embodiments of the invention provide a conjugate having the structure of formula (III):

P—L-cargo (III)

wherein:
P is a peptidyl residue of the macrocyclic compound described herein;
cargo is a chemical or biological agent (e.g., a detectable agent, synthetic biodegradable polymer, peptide, polypeptide, or polynucleotide); and
L is a linking moiety.

Certain embodiments of the invention provide a conjugate having the structure of formula (IV)

ₘ(P—L -Cargo [symbol] (IV)

wherein:
each P is independently a peptidyl residue of the macrocyclic compound described herein;
cargo is a biological agent (e.g., a synthetic biodegradable polymer, polypeptide, or polynucleotide);
each L is independently a linking moiety; and
m is an integer that is >=1.

Certain embodiments of the invention provide a conjugate having the structure of formula (V)

P—L [symbol] Cargo)ₙ (v)

wherein:
P is a peptidyl residue of the macrocyclic compound described herein;
each cargo is independently a chemical or biological agent (e.g., a detectable agent, synthetic biodegradable polymer, peptide, polypeptide, or polynucleotide);
L is a linking moiety; and
n is an integer that is >=1.

Certain embodiments of the invention provide a method for intracellular delivery, comprising contacting a cell with the macrocyclic compound as described herein or the conjugate as described herein.

Certain embodiments of the invention provide a method for treating a disease or condition, comprising administering a conjugate described herein to a subject in need thereof, wherein the conjugate comprises a cargo that is a therapeutic agent.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1F. Hydroxyl-rich cyclic peptides facilitate the transportation of cargo molecules into cells. (FIG. 1a) Generic structure of the EPPs. (FIG. 1b) Sequences of EPP1-7. (FIG. 1c) General method for assessing EPP uptake. (FIG. 1d) Confocal images showing that EPPs were able to bring rhodamine B (RB) tag into U87 cells with different efficiencies. The scale bar shows 50 μm. The bar graph shows the single-cell intracellular fluorescence intensities quantified from the confocal images. The error bars denote the standard deviation. RB-TAT was used for comparison. (FIG. 1e) Confocal images showing that EPP6 was able to bring different cargo molecules into the cells. The scale bar shows 50 μm. (FIG. 1f) Confocal images showing that RB-EPP6 was taken up by a wide panel of cell lines. The scale bar shows 50 μm.

FIGS. 2A-2D. EPP6 entered the cell through an energy-dependent process. (FIG. 2a) Illustration of the octanol partitioning experiment. (FIG. 2b) Results of the octanol partitioning experiments. Most of the EPPs showed a positive logP value. (FIG. 2c) Illustration of the process for comparing RB-EPP6 uptake at different temperatures. (FIG. 2d) Confocal images showing that low-temperature incubate led to no RB-EPP6 uptake in U87 cells. The scale bar shows 50 μm.

FIGS. 3A-3C. EPP6 enters the cells through a dynamin- and caveolin-dependent endocytosis pathway. (FIG. 3a) Illustration of the endocytosis inhibitor tests. (FIG. 3b) Flow cytometry results showing the effects of inhibitors on the uptake of RB-EPP6 in U87 cells. (FIG. 3c) Representative confocal images showing the overlap between mEmerald-caveolin and RB-EPP6. No significant colocalization was observed between mEmerald-clathrin and RB-EPP6. The scale bar shows 10 μm. CRISPR caveolin caused decreased RB-EPP6 uptake in U87 cells (data not shown).

FIGS. 4A-4D. FIBCD1 is a surface receptor for EPP6 recognition. (FIG. 4a) Illustration of the sample preparation for RNA-seq. (FIG. 4b) RNA-seq results identifying the FIBCD1 gene as the potential receptor for EPP6. The red dot in the graph represents the FIBCD1 gene. (FIG. 4c) Illustration of the process for comparing RB-EPP6 uptake at the presence of different FIBCD1 ligands. (FIG. 4d) Flow cytometry results showing the effects of FIBCD1 ligands on the uptake of RB-EPP6 in U87 cells.

FIGS. 5A-5D. Intracellular fate of EPP6. (FIG. 5a) Representative confocal images showing the overlap between EGFP-Rab5 and RB-EPP6 at 15 min. Significant colocalization was observed between mEmerald-Rab7a and RB-EPP6 starting at 30 min, the colocalization lasted for up to 4 hours. (FIG. 5b) Representative confocal images showing overlaps between LysoTracker and RB-EPP6. The scale bar shows 10 μm in the original images on the left and 2 μm in the zoomed in images on the right. (FIG. 5c) Representative confocal images showing RB-EPP6 signals in U87 cells at a time-dependence manner. The scale bar shows 10 μm. (FIG. 5d) The bar graph showing the Papp values for RB-EPP6 and RB-TAT.

FIGS. 6A-6C. Concentration-dependent uptake of RB-EPP6. (FIG. 6a) Illustration of the incubation process of RB-EPP6 at different concentrations in U87 cells. (FIG. 6b) Confocal images showing the uptake of RB-EPP6 by U87 cells increased as the concentration of RB-EPP6 gets higher. (FIG. 6c) The line chart shows the single-cell intracellular fluorescence intensities quantified from the confocal images.

FIGS. 7A-7B. (FIG. 7a) PAMPA assay results of EPPs. (FIG. 7b) The bar graph shows PAMPA value for each EPP quantified from the table on the left.

FIG. 8. Lipid binding assay results indicating no significant interaction between lipid components and RB-EPP6.

FIG. 9. Heparin binding assay. The bar graph shows that heparin exhibited no inhibitory effect towards RB-EPP6 uptake in U87 cells quantified from flow cytometry.

FIG. 10. Representative confocal images showing overlaps between different trackers and RB-EPP6, ERTracker (upper) and MitoTracker (lower). The scale bar shows 10 μm in the original images on the left and 2 μm in the zoomed in images on the right.

FIGS. 11A-11C. Confocal Images of U87 cells. FIG. 11A. Hoechst Dye Only. FIG. 11B. EPP6-RB with Hoechst. FIG. 11C. EPP6-GEM with Hoechst.

FIGS. 12A-12C. Crystal Violet Images of EPP6-GEM in U87 cells. FIG. 12A. no treatment, FIG. 12B. 500 nM EPP6-GEM, FIG. 12C. 500 nM GEM.

FIGS. 13A-13C. Crystal Violet Images of EPP6-GEM in FIBCD1-KO U87 cells. FIG. 13A. no treatment, FIG. 13B. 500 nM EPP6-GEM, FIG. 13C. 500 nM GEM.

FIG. 14. MALDI spectrum of EPP6-Insulin Conjugate mass which is indicated on the right side of the spectrum m/z=6928.

### DETAILED DESCRIPTION

The invention described herein relates to cell penetrating peptide (CPP) compounds. In certain embodiments, the macrocyclic compounds described herein may enter cells via endocytosis mediated transport. Thus, in certain embodiments, the invention described herein may also relate to endocytosis-promoting peptide (EPP) compounds.

As described herein, a positive charge may not be indispensable to confer a cell penetrating property to a CPP compound. Accordingly, in certain embodiments, the macrocyclic compounds described herein are CPP compounds free of a positive charge.

In certain embodiments, the macrocyclic compounds (e.g., cell penetrating peptide (CPP) compounds) described herein contain one or more hydroxy groups (e.g., 2, 3, 4, 5, 6 or more hydroxy groups). As discussed herein, hydroxy rich hydrophilic CPP compounds described herein may bind fibrinogen C domain-containing protein 1 (FIBCD1). Accordingly, in certain embodiments, the macrocyclic compound described herein is a FIBCD1 targeting agent.

### Peptide Compounds

Certain embodiments of the invention provide a macrocyclic compound (e.g., cell penetrating peptide (CPP) compounds) comprising a pentapeptide segment X₁-X₂-X₃-X₄-X₅, wherein:

X₁, X₂, X₃, X₄, and X₅ are each independently a residue of Phe, Tyr, Thr, Ser, or homo-Ser;

wherein Phe or Tyr is optionally substituted with one or more halo or hydroxy group on the phenyl ring; and

wherein Thr, Ser, or homo-Ser is optionally substituted with one or more halo, hydroxy, (C₁-C₆)alkyl, (C₃-C₆)cycloalkyl, aryl, or heteroaryl, wherein the (C₁-C₆)alkyl, (C₃-C₆)cycloalkyl, aryl, or heteroaryl is optionally substituted with one or more halo or hydroxy groups;

or a peptidyl residue or a salt thereof.

As described herein, in certain embodiments, a macrocyclic compound as described herein does not comprise the pentapeptide segment Tyr-Tyr-Thr-Tyr-Thr (SEQ ID NO:1).

In certain embodiments, the macrocyclic compound is a compound with molecular weight less than 5,000 Dalton. In certain embodiments, the macrocyclic compound is a compound with molecular weight less than 4,000 Dalton. In certain embodiments, the macrocyclic compound is a compound with molecular weight less than 3,000 Dalton. In certain embodiments, the macrocyclic compound is a compound with molecular weight less than 2,500 Dalton. In certain embodiments, the macrocyclic compound is a compound with molecular weight less than 2,000 Dalton. In certain embodiments, the macrocyclic compound is a compound with molecular weight less than 1,500 Dalton. In certain embodiments, the macrocyclic compound is a compound with molecular weight less than 1,000 Dalton. In certain embodiments, the macrocyclic compound is a small molecular compound with molecular weight less than 1,000 g/mol.

The term “peptide” describes a sequence of 5 to 25 amino acids (e.g., as defined herein) or its peptidyl residues. In certain embodiments, a peptide described herein comprises 6 to 20, or 7 to 15 amino acids. In certain embodiments, a peptide described herein comprises 5 to 16, or 7 to 10 amino acids. In certain embodiments, a peptide described herein comprises 5 to 12, or 7 to 9 amino acids. Peptide derivatives can be prepared as disclosed in U.S. Pat. Nos. 4,612,302; 4,853,371; and 4,684,620, or as described in the Examples herein below. Peptide sequences specifically recited herein are written with the amino terminus on the left and the carboxy terminus on the right. For example, a cyclic peptide described herein can be prepared using a variety of suitable cyclization methods (e.g., via click chemistry as described herein and in Example 1).

As used herein, the term “residue of an amino acid” means an amino acid wherein one or more atoms (e.g., H or OH) have been removed to provide an open valence that is used to link the amino acid to form a peptide bond, or to link to a carboxy-terminal group (e.g., to form an amidated C-terminal) or to link to an amino-terminus group (e.g., to form an acylated N-terminal).

As used herein, the term “peptidyl residue” means a peptide wherein one or more atoms (e.g., H or OH) have been removed to provide an open valence that is used to link the peptide to another compound or moiety, for example, to form a covalent bond with another compound or moiety. For example, a peptidyl residue of a CPP compound described herein could be the targeting and/or cell penetrating moiety in a conjugate comprising the moiety and a cargo compound (e.g., a fluorescent dye to be delivered into a cell).

In one embodiment, the peptidyl residue is a peptide compound wherein one or more atoms (e.g., H) have been removed from its N-terminal amine or a side chain amine of an amino acid residue (e.g., Lys) so to provide an open valence of —NH— that is used to link the peptide to another cargo compound, for example, via an amide bond.

In one embodiment, the peptidyl residue is a peptide compound wherein one or more atoms (e.g., H or OH) have been removed from its C-terminal or a side chain carboxy of an amino acid residue so to provide an open valence of —C(═O)—O or —C(═O)— that is used to link the peptide to another cargo compound, for example, via an ester or amide bond.

In one embodiment, the peptidyl residue is a peptide compound wherein one or more atoms (e.g., H or OH) have been removed from a mercapto group so to provide an open valence of —S— that is used to link the peptide to another cargo compound, for example, via a disulfide, thioether or thioester bond.

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., homo-Ser, 3,4-Dihydroxy-phenylalanine, 3-(3,4-Dihydroxyphenyl)serine, 2-Fluoro-5-hydroxy-tyrosine, 3-(2,4,5-Trihydroxyphenyl) alanine, 3,5-diiodo-tyrosine, 3,5-Dibromotyrosine, (3I)Tyr, 2-Hydroxyphenylalanine, meta-Tyrosine, 3-Chlorotyrosine, 4-hydroxy-threonine, Fluorophenylalanine, Pentafluorophenylalanine, Propargylglycine (Pra), Azidolysine (Az4), N-Methyl-proline, 2-Methylproline, 3-Methyl-histidine, 1-Methyl-histidine, 5-Fluoro-tryptophan, octenylalanine, pentenylalanine, pentenylglycine, azido-pentanoic acid, azidohomoalanine, azidophenylalanine, 5-Azido-2-amino-pentanoic acid, 2-Azido-3-phenylpropionic acid, 2-amino-5-hexynoic acid, 2-Azido-3-(3-indolyl)propionic acid, (pCl)Phe, ε-Aminocaproic acid, norleucine (Nle), para-I-phenylalanine ((pI)Phe), 2-napthylalanine (2-Nal), β-cyclohexylalanine (Cha), β-alanine ((β-Ala), gamma-carboxyglutamate, hippuric acid, octahydroindole-2-carboxylic acid, 1,2,3,4,-tetrahydroisoquinoline-3-carboxylic acid (Tic), penicillamine, ornithine, citruline, α-methyl-alanine, sarcosine, and tert-butylglycine) in D or L form. The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein).

As used herein, the term “cell penetrating” refers to the ability to enter a cell and/or to cross a cell membrane. The term “cell penetrating peptide” or CPP refers to a peptide as defined herein with the ability to enter a cell and/or to a cross cell membrane. The term CPP compound refers to a macrocyclic compound comprising a peptide segment as defined herein, with the ability to enter a cell and/or to cross a cell membrane.

In certain embodiments, the pentapeptide segment X₁-X₂-X₃-X₄-X₅ comprises one or more hydroxy groups. For example, the pentapeptide segment X₁-X₂-X₃-X₄-X₅ comprises one or more hydroxy groups on the side chain(s) of amino acid residue(s) within the pentapeptide segment.

In certain embodiments, the pentapeptide segment X₁-X₂-X₃-X₄-X₅ comprises two hydroxy groups. In certain embodiments, the pentapeptide segment X₁-X₂-X₃-X₄-X₅ comprises three hydroxy groups. In certain embodiments, the pentapeptide segment X₁-X₂-X₃-X₄-X₅ comprises four hydroxy groups. In certain embodiments, the pentapeptide segment X₁-X₂-X₃-X₄-X₅ comprises five hydroxy groups.

In certain embodiments, the peptide segment of the macrocyclic compound (e.g., the pentapeptide segment) is free of positively charged group on the side chains of the amino acid residues.

### X₁ Residue

In certain embodiments, X₁ is a residue of Phe or Tyr that is optionally substituted with one or more halo or hydroxy groups on the phenyl ring.

In certain embodiments, X₁ is a residue of Phe or Tyr.

In certain embodiments, X₁ is a residue of Tyr.

In certain embodiments, X₁ is a residue of Ser.

In certain embodiments, X₁ is a residue of Thr.

### X₂ Residue

In certain embodiments, X₂ is a residue of Thr, Ser, or homo-Ser that is optionally substituted with halo, hydroxy, (C₁-C₆)alkyl, (C₃-C₆)cycloalkyl, aryl, or heteroaryl, wherein the (C₁-C₆)alkyl, (C₃-C₆)cycloalkyl, aryl, or heteroaryl is optionally substituted with one or more halo or hydroxy groups.

In certain embodiments, X₂ is a residue of Thr, Ser, or homo-Ser.

In certain embodiments, X₂ is a residue of Thr.

In certain embodiments, X₂ is a residue of Ser.

In certain embodiments, X₂ is a residue of homo-Ser.

In certain embodiments, X₂ is a residue of Tyr.

### X₃ Residue

In certain embodiments, X₃ is a residue of Phe, Tyr, Thr, Ser, or homo-Ser.

In certain embodiments, X₃ is a residue of Phe or Tyr that is optionally substituted with one or more halo or hydroxy group on the phenyl ring.

In certain embodiments, X₃ is a residue of Phe or Tyr.

In certain embodiments, X₃ is a residue of Tyr.

In certain embodiments, X₃ is a residue of Thr, Ser, or homo-Ser that is optionally substituted with halo, hydroxy, (C₁-C₆)alkyl, (C₃-C₆)cycloalkyl, aryl, or heteroaryl, wherein the (C₁-C₆)alkyl, (C₃-C₆)cycloalkyl, aryl, or heteroaryl is optionally substituted with one or more halo or hydroxy groups.

In certain embodiments, X₃ is a residue of a residue of Thr, Ser, or homo-Ser.

In certain embodiments, X₃ is a residue of Thr.

In certain embodiments, X₃ is a residue of Ser.

In certain embodiments, X₃ is a residue of homo-Ser.

### X₄ Residue

In certain embodiments, X₄ is a residue of Phe or Tyr that is optionally substituted with one or more halo or hydroxy groups on the phenyl ring.

In certain embodiments, X₄ is a residue of Phe or Tyr.

In certain embodiments, X₄ is a residue of Tyr.

In certain embodiments, X₄ is a residue of Thr.

In certain embodiments, X₄ is a residue of Ser.

### X₅ Residue

In certain embodiments, X₅ is a residue of Thr, Ser, or homo-Ser that is optionally substituted with halo, hydroxy, (C₁-C₆)alkyl, (C₃-C₆)cycloalkyl, aryl, or heteroaryl, wherein the (C₁-C₆)alkyl, (C₃-C₆)cycloalkyl, aryl, or heteroaryl is optionally substituted with one or more halo or hydroxy groups.

In certain embodiments, X₅ is a residue of Thr, Ser, or homo-Ser.

In certain embodiments, X₅ is a residue of Thr.

In certain embodiments, X₅ is a residue of Ser.

In certain embodiments, X₅ is a residue of homo-Ser.

In certain embodiments, X₅ is a residue of Tyr.

Exemplary Pentapeptide Segment X₁-X₂-X₃-X₄-X₅

In certain embodiments, X₁ and X₄ are each independently a residue of Phe or Tyr that is optionally substituted with one or more halo or hydroxy groups on the phenyl ring.

In certain embodiments, X₁ and X₄ are each independently a residue of Phe or Tyr.

In certain embodiments, X₁ and X₄ are each a residue of Tyr.

In certain embodiments, X₁, X₃ and X₄ are each independently a residue of Phe or Tyr that is optionally substituted with one or more halo or hydroxy groups on the phenyl ring.

In certain embodiments, X₁, X₃ and X₄ are each independently a residue of Phe or Tyr.

In certain embodiments, X₁, X₃ and X₄ are each a residue of Tyr.

In certain embodiments, X₂ and X₅ are each independently a residue of Thr, Ser, or homo-Ser that is optionally substituted with halo, hydroxy, (C₁-C₆)alkyl, (C₃-C₆)cycloalkyl, aryl, or heteroaryl, wherein the (C₁-C₆)alkyl, (C₃-C₆)cycloalkyl, aryl, or heteroaryl is optionally substituted with one or more halo or hydroxy groups.

In certain embodiments, X₂ and X₅ are each independently a residue of Thr, Ser, or homo-Ser.

In certain embodiments, X₂ and X₅ are each independently a residue of Thr, or Ser.

In certain embodiments, X₁ and X₄ are each independently a residue of Phe or Tyr; and X₂ and X₅ are each independently a residue of Thr, Ser, or homo-Ser.

In certain embodiments, X₁ and X₄ are each independently a residue of Tyr; and X₂ and X₅ are each independently a residue of Thr, Ser, or homo-Ser.

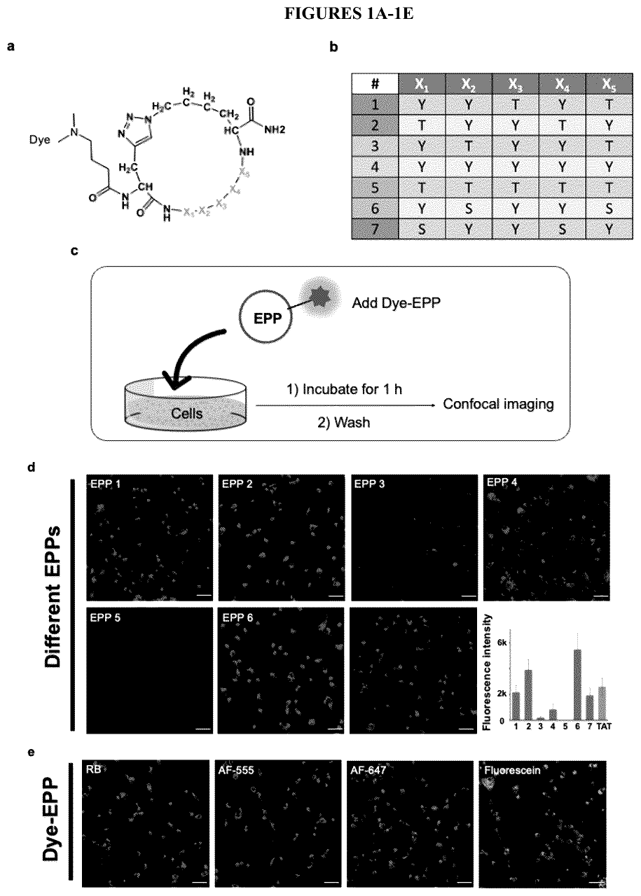

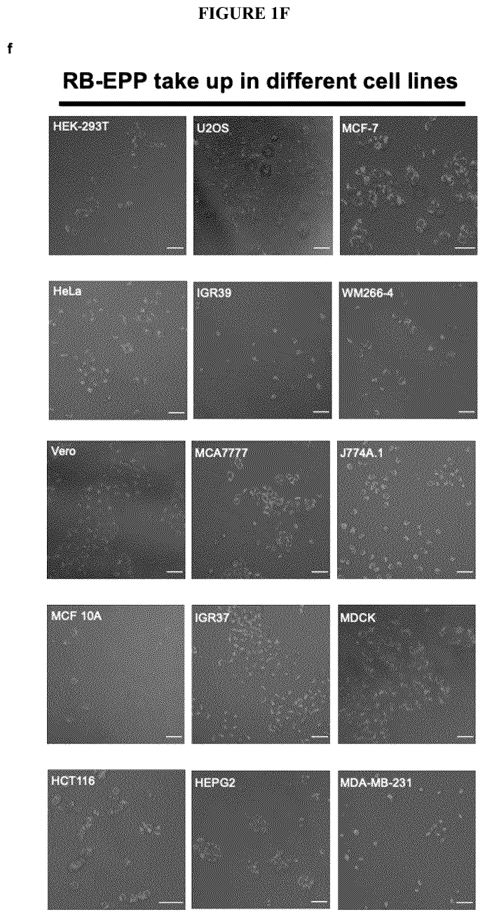

In certain embodiments, the pentapeptide segment is a sequence shown in FIG. 1b.

In certain embodiments, the pentapeptide segment is selected from the group consisting of

(SEQ ID NO: 2)
Thr-Tyr-Tyr-Thr-Tyr,
(SEQ ID NO: 3)
Tyr-Thr-Tyr-Tyr-Thr,
(SEQ ID NO: 4)
Tyr-Tyr-Tyr-Tyr-Tyr,
(SEQ ID NO: 6)
Tyr-Ser-Tyr-Tyr-Ser,
and
(SEQ ID NO: 7)
Ser-Tyr-Tyr-Ser-Tyr.

In certain embodiments, the pentapeptide segment is Thr-Tyr-Tyr-Thr-Tyr (TYYTY, SEQ ID NO:2).

In certain embodiments, the pentapeptide segment is Tyr-Thr-Tyr-Tyr-Thr (YTYYT, SEQ ID NO:3).

In certain embodiments, the pentapeptide segment is Tyr-Tyr-Tyr-Tyr-Tyr (YYYYY, SEQ ID NO:4).

In certain embodiments, the pentapeptide segment is Tyr-Ser-Tyr-Tyr-Ser (YSYYS, SEQ ID NO:6).

In certain embodiments, the pentapeptide segment is Ser-Tyr-Tyr-Ser-Tyr (SYYSY, SEQ ID NO:7).

### Exemplary Macrocyclic Compounds

As used herein, the term “macrocycle compound” or “macrocyclic compound” refers to a cyclic compound comprising a peptide segment (e.g., a pentapeptide segment X₁-X₂-X₃-X₄-X₅). In one embodiment, the macrocyclic compound comprises 15-50 atoms linked by peptide or non-peptide bonds to form a ring. In one embodiment, the macrocyclic compound comprises 15-40 atoms linked by peptide or non-peptide bonds to form a ring. In one embodiment, the macrocyclic compound comprises 15-30 atoms linked by peptide or non-peptide bonds to form a ring. In one embodiment, the macrocyclic compound comprises 15-25 atoms linked by peptide or non-peptide bonds to form a ring. In one embodiment, the macrocyclic compound comprises 20-50 atoms linked by peptide or non-peptide bonds to form a ring. In one embodiment, the macrocyclic compound comprises 30-50 atoms linked by peptide or non-peptide bonds to form a ring. In one embodiment, the macrocyclic compound comprises 40-50 atoms linked by peptide or non-peptide bonds to form a ring. In one embodiment, the macrocyclic compound comprises 20-40 atoms linked by peptide or non-peptide bonds to form a ring. In one embodiment, the macrocyclic compound comprises 20-30 atoms linked by peptide or non-peptide bonds to form a ring. The designation c[] as used herein, for example c[X₀-X₁-X₂-X₃-X₄-X₅-X₆], designates a cyclic peptide sequence, e.g., cyclic X₁-X₁-X₂-X₃-X₄-X₅-X₆, wherein the residue X₀ and the residue X₆ are cyclized.

In certain embodiments, the macrocycle compound comprises a peptide segment of 5 to 25 amino acids. For example, in certain embodiments, the macrocycle compound is a cyclic peptide. The macrocycle could be formed via a variety of cyclization modes (e.g., via peptide bond or non-peptide bond cyclization as described herein). Any two natural and/or unnatural amino acid (AA) residues (e.g., terminal residues) can form a covalent bond to achieve cyclization. For example, two AA residues of a peptide can cyclize via head to tail (N terminal to C terminal), sidechain to sidechain (e.g., disulfide bond, or via click chemistry as shown in Example 1), head to sidechain, or sidechain to tail. The cyclization bond and chemistry can be through the formation of, e.g., an amide bond, disulfide bond, ether bond, thiol-ether bond, lactone, or “click” chemistry (e.g., via azide-alkyne “click” chemistry) between any two natural and/or unnatural amino acids.

In certain embodiments, the compound is a macrocycle compound (e.g., cyclic peptide) comprising a peptide segment having 5 to 12 (e.g., 5, 6, 7, 8, 9, 10, 11, or 12) amino acids (aa) in length, or a peptidyl residue thereof, or a salt thereof. In certain embodiments, the peptide segment has 5-12, 6-12, or 7-12 aa in length. In certain embodiments, the peptide segment has 5-10, 5-9, or 5-8 aa in length. In certain embodiments, the peptide segment has 5 to 9 (e.g., 5, 6, 7, 8, 9) aa in length. In certain embodiments, the peptide segment has 5-11, 6-10, or 7-9 aa in length. In certain embodiments, the peptide segment has at least 6, 7, 8, 9, 10 or 11 aa in length.

In certain embodiments, a macrocyclic compound is cyclized via sidechain to sidechain cyclization mode. For example, in certain embodiments, a macrocyclic compound comprising a peptide sequence X₀-X₁-X₂-X₃-X₄-X₅-X₆ (from N terminal to C terminal) is cyclized via the sidechain of residue X₀ and the sidechain of residue X₆, wherein the N terminal (e.g., NH₂—) at X₀ and the C terminal (e.g., —COOH) at X₆ do not participate in the cyclization.

In certain embodiments, the macrocyclic compound, from N terminal to C terminal, has formula I:

(Rₙ)₂N-c[X₀-X₁-X₂-X₃-X₄-X₅-X₆]—C(═O)-R_{c} (I)

wherein:

X₀ is a residue of an amino acid,

X₆ is a residue of an amino acid,

X₁-X₂-X₃-X₄-X₅ is the pentapeptide segment as described herein,

each Rₙ is independently H, (C₁-C₆)alkyl, or (C₁-C₆)alkanoyl, and

R_{c} is hydroxy or —N(R_{f})₂, wherein each R_{f} is independently H or (C₁-C₆)alkyl.

In certain embodiments, Rₙ are each H.

In certain embodiments, R_{f} are each H.

In certain embodiments, R_{c} is hydroxy.

In one embodiment of the invention, the peptide is cyclized via two direct bonds formed through click chemistry between a residue of an amino acid that has an alkynyl group and another residue of an amino acid that has an azido group, wherein the alkynyl group and the azido group formed two direct bonds in a triazole group.

In one embodiment, the residue of an amino acid that has an alkynyl group is a residue of Pra, and the residue of an amino acid that has an azido group is a residue of Az4. The compound is cyclized through click chemistry between alkynyl group of Pra and azido group of Az4, resulting in a triazole group that contains two direct bonds formed between the residue of Pra and the residue of Az4.

It is to be understood that there are a wide range of scaffolds and non-amino acid linkers known to person skilled in the art that may be used to cyclize a peptide or compound described herein (see, e.g., Donghyeok Gang, et al, Genes. 2018 November; 9(11): 557 and YH Lau, at al, Chem Soc Rev. 2015 Jan. 7;44(1):91-102). In certain embodiments, X₀ or X₆is a residue of an amino acid that has an alkynyl group on its side chain, and X₆ or X₀ is a residue of an amino acid that has an azido group on its side chain.

In certain embodiments, X₀ or X₆ is L-Pra or D-Pra. In certain embodiments, X₆ or X₀ is L-Az4, D-Az4, L-azido-pentanoic acid, D-azido-pentanoic acid, L-azidohomoalanine, D-azidohomoalanine, L-azidophenylalanine, or D-azidophenylalanine.

In certain embodiments, X₀ is a residue of Pra and X₆is a residue of Az4. In certain embodiments, X₆ is a residue of Pra and X₀ is a residue of Az4.

In certain embodiments, the cyclic peptide sequence is a sequence described in FIG. 1 (see Example 1).

In certain embodiments, the macrocyclic compound comprises a cyclic peptide sequence selected from the group consisting of

(SEQ ID NO: 9)
c[Pra-Thr-Tyr-Tyr-Thr-Tyr-Az4],
(SEQ ID NO: 10)
c[Pra-Tyr-Thr-Tyr-Tyr-Thr-Az4],
(SEQ ID NO: 11)
c[Pra-Tyr-Tyr-Tyr-Tyr-Tyr-Az4],
(SEQ ID NO: 13)
c[Pra-Tyr-Ser-Tyr-Tyr-Ser-Az4],
and
(SEQ ID NO: 14)
c[Pra-Ser-Tyr-Tyr-Ser-Tyr-Az4]

In certain embodiments, the macrocyclic compound comprises a cyclic peptide sequence c[Pra-Thr-Tyr-Tyr-Thr-Tyr-Az4] (SEQ ID NO:9).

In certain embodiments, the macrocyclic compound comprises a cyclic peptide sequence c[Pra-Tyr-Thr-Tyr-Tyr-Thr-Az4] (SEQ ID NO:10).

In certain embodiments, the macrocyclic compound comprises a cyclic peptide sequence c[Pra-Tyr-Tyr-Tyr-Tyr-Tyr-Az4] (SEQ ID NO:11).

In certain embodiments, the macrocyclic compound comprises a cyclic peptide sequence c[Pra-Tyr-Ser-Tyr-Tyr-Ser-Az4] (SEQ ID NO:13).

In certain embodiments, the macrocyclic compound comprises a cyclic peptide sequence c[Pra-Ser-Tyr-Tyr-Ser-Tyr-Az4] (SEQ ID NO:14).

In certain embodiments, the macrocyclic compound described herein has formula II:

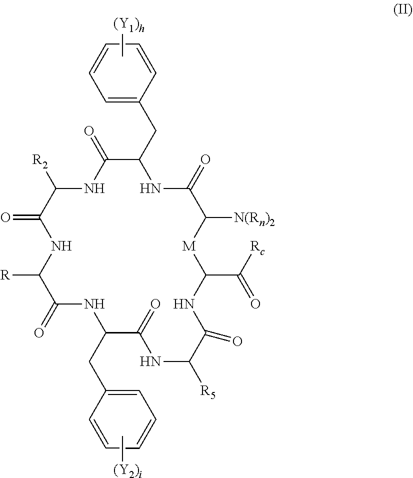

wherein
R₂ and R₅ are each independently (C₁-C₃)alkyl substituted with one or more hydroxy groups,
R is benzyl optionally substituted with one or more halo or hydroxy groups on the phenyl ring, or (C₁-C₃)alkyl substituted with one or more hydroxy groups,
each Y₁ is independently halo or hydroxy,
each Y₂ is independently halo or hydroxy,
h and i are each independently 0, 1, 2, 3, 4, or 5,
each Rₙ is independently H, (C₁-C₆)alkyl, or (C₁-C₆)alkanoyl,
R_{c} is hydroxy or —N(R_{f})₂,
each R_{f} is independently H or (C₁-C₆)alkyl,
M is divalent, branched or unbranched, saturated or unsaturated, hydrocarbon chain having from 2 to 16 carbon atoms, wherein one or more of the carbon atoms is optionally replaced by (—O—), (—S—), (—NRₐ—), (C₃-C₈)cycloalkyl, aryl, or heteroaryl (e.g., divalent, 6-10 membered aryl or 5-10 membered heteroaryl such as triazolyl), wherein the chain is optionally substituted on carbon with one or more substituents selected from the group consisting of halo, hydroxy, mercapto, oxo (═O), and thioxo(═O); wherein Rₐ is a H or (C₁-C₆)alkyl.

In certain embodiments, R₂ and R₅ are each independently (C₁-C₂)alkyl substituted with one or more hydroxy groups.

In certain embodiments, R₂ is hydroxymethyl, 1-hydroxyethyl, or 2-hydroxyethyl.

In certain embodiments, R₅ is hydroxymethyl, 1-hydroxyethyl, or 2-hydroxyethyl.

In certain embodiments, R is (C₁-C₂)alkyl substituted with one or more hydroxy groups.

In certain embodiments, R is hydroxymethyl, 1-hydroxyethyl, or 2-hydroxyethyl.

In certain embodiments, Y₁, and Y₂ are each hydroxy.

In certain embodiments, h and i are each independently 0, 1, 2, or 3.

In certain embodiments, Rₐ are each H.

In certain embodiments, R_{f} are each H.

In certain embodiments, the macrocyclic compound described herein has formula IIa:

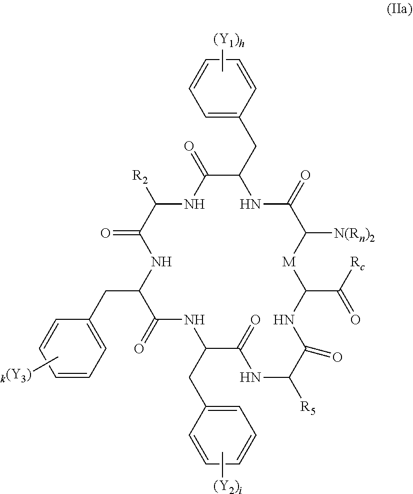

wherein each Y₃ is independently halo or hydroxy, and k is 0, 1, 2, 3, 4, or 5.

In certain embodiments, Y₁, Y₂, and Y₃ are each hydroxy.

In certain embodiments, h, i, and k are each independently 0, 1, 2, or 3.

In certain embodiments, the macrocyclic compound described herein has formula IIb:

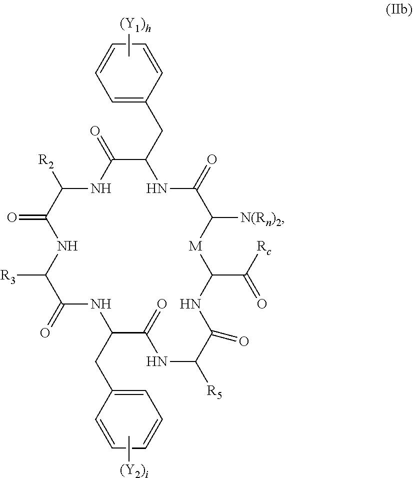

wherein R₃ is (C₁-C₃)alkyl substituted with one or more hydroxy groups.

In certain embodiments, R₃ is (C₁-C₂)alkyl substituted with one or more hydroxy groups.

In certain embodiments, R₃ is hydroxymethyl, 1-hydroxyethyl, or 2-hydroxyethyl.

In certain embodiments, the macrocyclic compound comprises a cyclic peptide sequence of

(SEQ ID NO: 10)
c[Pra-Tyr-Thr-Tyr-Tyr-Thr-Az4]
or
(SEQ ID NO: 13)
c[Pra-Tyr-Ser-Tyr-Tyr-Ser-Az4].

In certain embodiments, the macrocyclic compound described herein has structure of

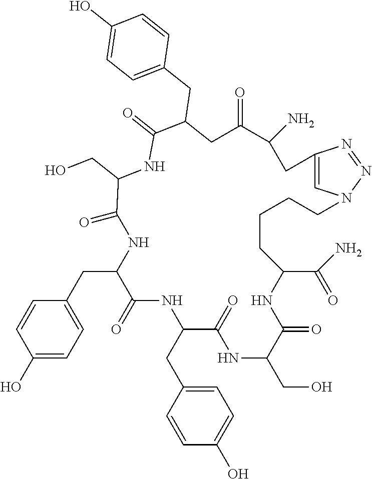 [SMILES: NC(=O)C1CCCCn2cc(nn2)CC(N)C(=O)CC(Cc2ccc(O)cc2)C(=O)NC(CO)C(=O)NC(Cc2ccc(O)cc2)C(=O)NC(Cc2ccc(O)cc2)C(=O)NC(CO)C(=O)N1]

In certain embodiments, the macrocyclic compound described herein is a cell penetrating peptide.

In certain embodiments, the macrocyclic compound described herein is capable of entering a cell via endocytosis.

In certain embodiments, the macrocyclic compound described herein is an endocytosis promoting peptide.

In certain embodiments, the macrocyclic compound is capable of entering a cell via caveolin and/or dynamin-dependent endocytosis. In certain embodiments, the macrocyclic compound is capable of entering a cell via caveolin-dependent endocytosis. In certain embodiments, the macrocyclic compound is capable of entering a cell via dynamin-dependent endocytosis. In certain embodiments, the macrocyclic compound is capable of entering a cell via lipid raft-independent endocytosis.

In certain embodiments, the macrocyclic compound is capable of binding human fibrinogen C domain-containing protein 1 (FIBCD1) (NCBI accession number NP 116232.3). In certain embodiments, the macrocyclic compound is capable of entering a cell via FIBCD1-mediated, caveolin and/or dynamin-dependent endocytosis.

### Conjugates

The macrocyclic compound described herein, or a peptidyl residue thereof, may be conjugated to a cargo. Thus, intracellular delivery of the cargo may be increased compared to a control cargo that is not conjugated with a macrocyclic compound described herein.

Accordingly, certain embodiments of the invention provide a conjugate having the structure of formula (III):

P—L-cargo (III)

wherein:
P is a peptidyl residue of the macrocyclic compound described herein;
cargo is a chemical or biological agent (e.g., a detectable agent, synthetic biodegradable polymer, peptide, polypeptide, or polynucleotide); and
L is a linking moiety;
or a salt thereof.

As used herein, the term “cargo” refers to a chemical or biological agent (e.g., a detectable agent (such as fluorescent dye or a radioactive agent), synthetic biodegradable polymer, peptide, polypeptide, or polynucleotide) to be delivered into a cell.

In certain embodiments, the cargo is a compound with molecular weight less than 5000 Dalton, 4000 Dalton, 3000 Dalton, or 2000 Dalton. In certain embodiments, cargo is a small molecule compound having molecular weight smaller than 1,000 g/mol. In certain embodiments, the cargo is a detectable agent (e.g., fluorescent dye moiety such as Rhodamine B, Thiazole Orange, Alexa Fluor 555 or Alexa Fluor 647, see Example 1; or a radioactive agent).

In certain embodiments, the cargo is negatively charged.

In certain embodiments, the cargo is a polymer.

As used herein, the term “polymer” refers to a molecule of repeating units (e.g., lactic acid, amino acids, or nucleotides) joined by repetitive bond (e.g., ester, amide, or phosphodiester bond). In certain embodiments, the polymer is a biodegradable polymer/copolymer, including but not limited to, polylactic acid (PLA), poly lactic-co-glycolic acid (PLGA), polycaprolactone (PCL). The term “polymer” used herein also includes polynucleotide or protein.

The terms “protein”, and “polypeptide” are used interchangeably herein. The term “protein” encompass a peptide as defined above and longer peptide of more than 25 amino acids in length. In certain embodiments, the term “protein” may refer to a single polypeptide or may refer to two or more polypeptides (e.g., dimerized or trimerized).

The term “polynucleotide” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid sequence or segment or fragment” or “polynucleotide” may be used interchangeably. These terms may also be used interchangeably with gene, cDNA, DNA and RNA encoded by a gene. In certain embodiments, the polynucleotide is a mRNA, siRNA, shRNA, or miRNA. The term also includes a modified nucleic acid molecule (e.g., phosphorothioate or phosphoramidite based polynucleotide).

In certain embodiments, the cargo is a detectable agent (e.g., fluorescent dye or radioactive agent).

In certain embodiments, the cargo is a therapeutic agent for treating a disease or condition.

In certain embodiments, the cargo is a therapeutic agent for treating cancer. In certain embodiments, the cargo is a therapeutic agent for treating solid tumor (e.g., bladder cancer, non-small cell lung cancer (NSCLC), breast cancer, ovarian cancer, cervical cancer, or pancreatic cancer). In certain embodiments, the cargo is a chemotherapeutic agent. In certain embodiments, the cargo is gemcitabine.

In certain embodiments, the cargo is a protein. In certain embodiments, the cargo is a protein that may be modified with any desirable moiety known to the one skilled in the art or described herein, for example, a non-limiting example of modified protein cargo may be a protein that is glycosylated, lipidated, pegylated, or labeled with fluorescent dye.

In certain embodiments, the cargo is a therapeutic agent for treating diabetes. In certain embodiments, the cargo is human insulin.

In certain embodiments, a conjugate has the structure of formula (III):

P—L-cargo (III)

wherein:
P is a peptidyl residue of a macrocyclic compound (a compound comprising a pentapeptide segment X₁-X₂-X₃-X₄-X₅) as described herein and L is a linking moiety.
In certain embodiments, L is a linking moiety having the structure:

—W—Z—T—
W is selected from the group consisting of a bond, —O—, —S—, —C(═O)—, —N(R_{S})—, —C (═O)NH—, —C(═S)NH—, —C(═O)O—, —C(═O)S—, —NHSO₂—, —OC(═O)NH—, —NHC(═O)NH—, and —NHC(═S)NH—, wherein R_{S} is H or (C₁-C₆)alkyl;
T is selected from the group consisting of a bond, —O—, —S—, —C(═O)—, —N(Rᵤ)—, —C (═O)NH—, —C(═S)NH—, —C(═O)O—, —C(═O)S—, —NHSO₂—, —OC(═O)NH—, —NHC(═O)NH—, and —NHC(═S)NH—, wherein R_{U} is H or (C₁-C₆)alkyl;
Z is selected from the group consisting of a bond, or a divalent, branched or unbranched, saturated or unsaturated, hydrocarbon chain having from 2 to 16 carbon atoms, wherein one or more of the carbon atoms is optionally replaced by (—O—), (—S—), (—NRₚ—), (C₃-C₈)cycloalkyl, aryl, or heteroaryl (e.g., divalent, 6-10 membered aryl or 5-10 membered heteroaryl such as triazolyl), wherein Rₚ is H or (C₁-C₆)alkyl, wherein the hydrocarbon chain is optionally substituted on carbon with one or more substituents selected from the group consisting of halo, hydroxy, mercapto, oxo(═O), and thioxo(═S).
In certain embodiments, L is a linking moiety having the structure:

—W—Z—T—
W is selected from the group consisting of a bond, —O—, —S—, —C(═O)—, —N(R_{S})—, —C (═O)NH—, —C(═S)NH—, —C(═O)O—, —C(═O)S—, —NHSO₂—, —OC(═O)NH—, —NHC(═O)NH—, and —NHC(═S)NH—, wherein R_{S} is H or (C₁-C₆)alkyl;
T is selected from the group consisting of a bond, —O—, —S—, —C(═O)—, —C (═O)NH—, —C(═S)NH—, —C(═O)O—, —C(═O)S—, —NHSO₂—, —OC(═O)NH—, —NHC(═O)NH—, and —NHC(═S)NH—, wherein Rᵤ is H or (C₁-C₆)alkyl;
Z is selected from the group consisting of a bond, or a divalent, branched or unbranched, saturated or unsaturated, hydrocarbon chain having from 2 to 16 carbon atoms, wherein one or more of the carbon atoms is optionally replaced by (—O—), (—S—), (—NRₚ—), (C₃-C₈)cycloalkyl, aryl, or heteroaryl (e.g., divalent, 6-10 membered aryl or 5-10 membered heteroaryl such as triazolyl), wherein Rₚ is H or (C₁-C₆)alkyl, wherein the hydrocarbon chain is optionally substituted on carbon with one or more substituents selected from the group consisting of halo, hydroxy, amino, mercapto, oxo(═O), and thioxo(═S).

In certain embodiments, the functional group of W or T (e.g., —C(═O)O—) may be oriented in one of both directions in formula (III). For example, in certain embodiments, ester of W or T may be oriented as —C(═O)O— or —OC(═O)—; amide of W or T may be oriented as —C(═O)NH— or —NH—C(═O)—. In certain embodiments, wherein Z is a bond, W and T are not heteroatoms at the same time.

In certain embodiments, the linker comprises disulfide bond (—S—S—), for example, two carbon atoms of Z are replaced by (—S—).

In certain embodiments, the linker has a molecular weight of from about 20 daltons to about 20,000 daltons.

In certain embodiments, the linker has a molecular weight of from about 20 daltons to about 10,000 daltons.

In certain embodiments, the linker has a molecular weight of from about 20 daltons to about 5,000 daltons.

In certain embodiments, the linker has a molecular weight of from about 20 daltons to about 3,000 daltons.

In certain embodiments, the linker has a molecular weight of from about 20 daltons to about 2,000 daltons.

In certain embodiments, the linker has a molecular weight of from about 20 daltons to about 1,000 daltons.

In certain embodiments, the linking moiety comprises a polyethylene glycol (PEG) group with formula —(OCH₂CH₂)ₘ—, wherein m is an integer from 2 to 120 (e.g., m is 24, or 43). For example, the PEG group may be a PEG (MW:1000), PEG (MW:2000) or PEG (MW:5000) chain.

In certain embodiments, the linker comprises one or more amino acid residue(s). In certain embodiments, the linking moiety comprises a lysine residue. In certain embodiments, the linking moiety comprises a cysteine residue.

In certain embodiments, the linker comprises or is:

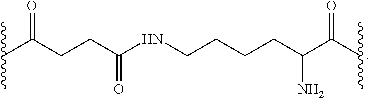 [SMILES: *C(=O)CCC(=O)NCCCCC(N)C(*)=O]

In certain embodiments, the linker comprises or is:

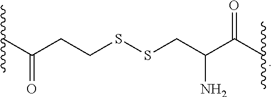 [SMILES: *C(=O)CCSSCC(N)C(*)=O]

In certain embodiments, N terminus of the macrocyclic compound (e.g., a cyclic peptide described herein) is conjugated to the cargo, for example, via an amide bond.

In certain embodiments, C terminus of the macrocyclic compound (e.g., a cyclic peptide described herein) is conjugated to the cargo, for example, via an amide or an ester bond.

In certain embodiments, the conjugate of formula (III) has structure of:

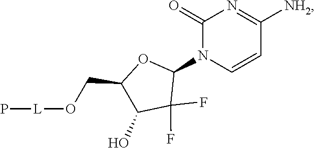

or a salt thereof.

In certain embodiments, P is a compound as described herein, such as compound 6 or peptidyl thereof.

Accordingly, in certain embodiments, the conjugate of formula (III) has structure of:

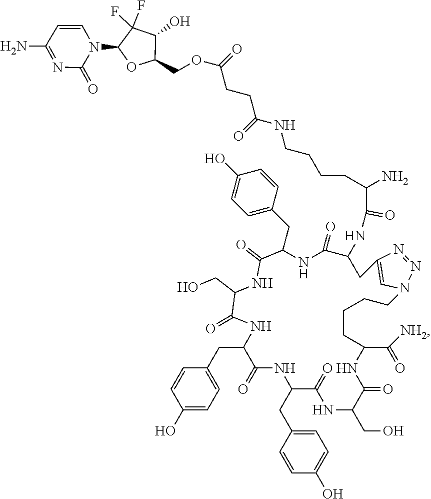 [SMILES: NC(=O)C1CCCCn2cc(nn2)CC(NC(=O)C(N)CCCCNC(=O)CCC(=O)OC[C@H]2O[C@@H](n3ccc(N)nc3=O)C(F)(F)[C@@H]2O)C(=O)NC(Cc2ccc(O)cc2)C(=O)NC(CO)C(=O)NC(Cc2ccc(O)cc2)C(=O)NC(Cc2ccc(O)cc2)C(=O)NC(CO)C(=O)N1]

or a salt thereof.

In certain embodiments, the conjugate of formula (III) has structure of:
P—L-insulin, or a salt thereof.

In certain embodiments, P is a compound as described herein, such as compound 6 or peptidyl thereof.

In certain embodiments, the conjugate of formula (III) has structure of:

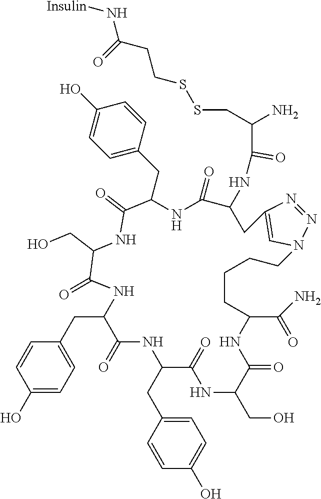

or a salt thereof.

In certain embodiments, the cargo is a protein. In certain embodiments, the cargo is a protein that may be modified with any desirable moiety known to art or described herein, such as a protein that is glycosylated, lipidated, pegylated, or labeled with fluorescent dye. For example, it is known to the one skilled in the art that a protein may have more than one amino acid residue side chain group that could be modified (e.g., amino group of lysine residues or thiol group of cysteine residues). Thus, when a cargo is a protein in a conjugate described herein (a conjugate of formula III above or formula IV below), it is to be understood that the cargo may be linked with one or more linker(s) and thus the cargo protein may be linked with one or more macrocyclic compound described herein via the linker(s).

For example, in certain embodiments, the invention also provides a conjugate of formula (IV) of

ₘ(P−L [symbol] Cargo (IV)

wherein:
each P is independently a peptidyl residue of the macrocyclic compound described herein;
cargo is a biological agent (e.g., a synthetic biodegradable polymer, polypeptide, or polynucleotide);
each L is independently a linking moiety; and
m is an integer that is >=1;
or a salt thereof.

In certain embodiments, the cargo is a protein (e.g., insulin).

In certain embodiments, m is 1. In certain embodiments, m is 1 or 2. In certain embodiments, m is 1, 2, or 3. In certain embodiments, m is 1, 2, 3, or 4. In certain embodiments, m is 1, 2, 3, 4, or 5. In certain embodiments, m is 1, 2, 3, 4, 5, or 6. In certain embodiments, m is 1, 2, 3, 4, 5, 6, or 7. In certain embodiments, m is 1, 2, 3, 4, 5, 6, 7, or 8.

Certain embodiments of the invention also provide an intermediate compound for making a conjugate described herein.

In certain embodiments, the intermediate compound is a peptide comprising the macrocyclic compound described herein (e.g., compound of Formula I or peptidyl thereof) and an amino acid residue (e.g., Lys or Cys). In certain embodiments, the amino acid residue (e.g., Lys or Cys) forms an amide bond with the N terminus of the macrocyclic compound. Thus, in certain embodiments, the intermediate compound has structure of Xₜ-HN-c[X₀-X₁-X₂-X₃-X₄-X₅-X₆]—C (═O)-R_{c}, wherein Xₜ is an amino acid residue (e.g., Lys or Cys), and other variables are as described herein in Formula I. In certain embodiments, the intermediate compound has structure:

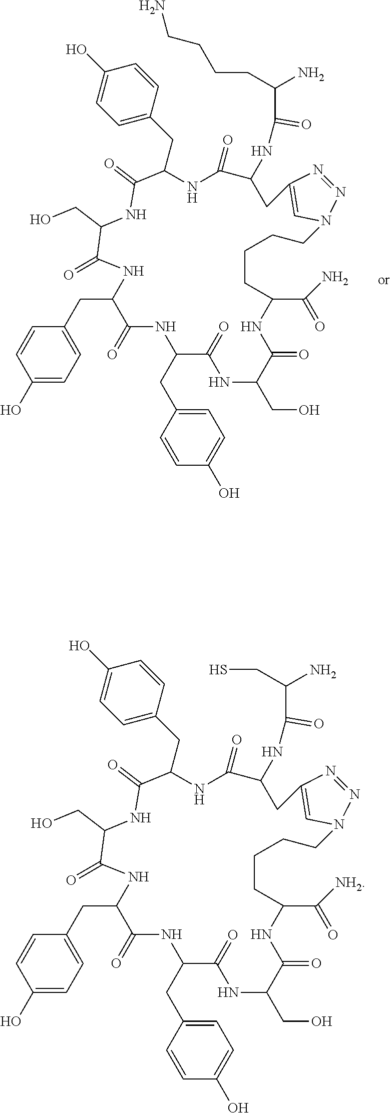 [SMILES: NCCCCC(N)C(=O)NC1Cc2cn(nn2)CCCCC(C(N)=O)NC(=O)C(CO)NC(=O)C(Cc2ccc(O)cc2)NC(=O)C(Cc2ccc(O)cc2)NC(=O)C(CO)NC(=O)C(Cc2ccc(O)cc2)NC1=O ; NC(=O)C1CCCCn2cc(nn2)CC(NC(=O)C(N)CS)C(=O)NC(Cc2ccc(O)cc2)C(=O)NC(CO)C(=O)NC(Cc2ccc(O)cc2)C(=O)NC(Cc2ccc(O)cc2)C(=O)NC(CO)C(=O)N1]

Certain embodiments of the invention provide a conjugate of formula (V) of

P−L [symbol] Cargo)ₙ (V)

wherein:
P is a peptidyl residue of the macrocyclic compound described herein;
each cargo is independently a chemical or biological agent (e.g., a detectable agent, synthetic biodegradable polymer, peptide, polypeptide, or polynucleotide); and
L is a linking moiety;
n is an integer that is >=1;
or a salt thereof.

In certain embodiments, n is 1. In certain embodiments, n is 1 or 2. In certain embodiments, n is 2. In certain embodiments, n is 1, 2, or 3.

In certain embodiments, the cargos are the same cargo (e.g., a therapeutic agent cargo).

In certain embodiments, the cargos are different cargos (e.g., a therapeutic agent cargo and a fluorescent dye cargo).

In certain embodiments, the conjugate of formula (V) has structure of

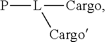

wherein the cargo and cargo' may be the same or different cargos.

In certain embodiments, the conjugate of formula (V) is

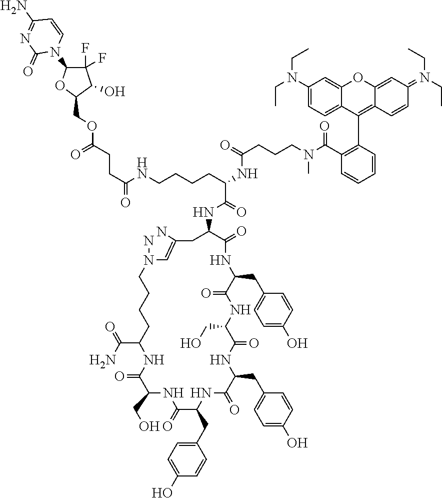

or salt thereof

### Methods

Certain embodiments of the invention provide a method for intracellular delivery, comprising contacting a cell with a macrocyclic compound (or a peptidyl residue thereof) or a conjugate described herein.

Certain embodiments of the invention provide a method for targeted delivery, comprising contacting a cell with a FIBCD1 targeting macrocyclic compound (or a peptidyl residue thereof) or a conjugate described herein.

In certain embodiments, the cell is contacted in vitro.

In certain embodiments, the cell is contacted in vivo.

In certain embodiments, the cell is a ciliated epithelial cell.

In certain embodiments, the cell is a ciliated epithelial cell in the digestive track (e.g., intestine) of an animal.

In certain embodiments, the macrocyclic compound or the conjugate is delivered to an endosome, lysosome, and/or cytosol of the cell.

In certain embodiments, the macrocyclic compound or the conjugate is delivered to an endosome of the cell.

In certain embodiments, the macrocyclic compound or the conjugate is delivered to a lysosome of the cell.

In certain embodiments, the macrocyclic compound or the conjugate is delivered to cytosol of the cell.

In certain embodiments, the macrocyclic compound or the conjugate enters the cell via endocytosis and exits the cell via exocytosis (i.e., capable of transcytosis).

Certain embodiments of the invention provide a method for treating a disease or condition, comprising administering a conjugate described herein to a mammal in need thereof, wherein the cargo is a therapeutic agent.

Certain embodiments provide a conjugate as described herein for use in treating a disease or condition in a mammal in need thereof, wherein the cargo is a therapeutic agent.

Certain embodiments provide the use of a conjugate as described herein in the preparation of a medicament for treating a disease or condition in a mammal in need thereof, wherein the cargo is a therapeutic agent.

Certain embodiments of the invention provide a diagnostic or therapeutic method comprising administering a conjugate described herein to a mammal, wherein the cargo is a detectable agent or a therapeutic agent.

Certain embodiments provide a conjugate as described herein for use in a diagnostic or therapeutic method, the method comprising administering the conjugate to a mammal in need thereof, wherein the cargo is a detectable agent or a therapeutic agent.

Certain embodiments provide the use of a conjugate as described herein in the preparation of a medicament for use in a diagnostic or therapeutic method.

In certain embodiments, the disease is diabetes. In certain embodiments, the disease is cancer. In certain embodiments, the disease is a solid tumor. In certain embodiments, the cancer is epithelial cancer. In certain embodiments, the cancer is bladder cancer, non-small cell lung cancer (NSCLC), breast cancer, ovarian cancer, cervical cancer, or pancreatic cancer.

Macrocyclic compounds and conjugates described herein (including a salt thereof) can be formulated in a composition. Certain embodiments of the invention provide a composition comprising a macrocyclic compound or conjugate described herein. In certain embodiments, the composition is in liquid form. In certain embodiments, the composition is in solid form. In certain embodiments, the composition is in lyophilized form.

Lyophilized formulations may also contain bulking agent (e.g., mannitol or glycine) and cryoprotectant/lyoprotectant (e.g., trehalose or sucrose). Lyophilized formulation can be reconstituted into a liquid dosage form using saline, 5% dextrose solution or sterile water before use. Certain embodiments of the invention provide a method of delivering a cargo to a FIBCD1 expressing cell that is present in an animal in need thereof (e.g., a cell in the digestive tract, such as an intestinal cell), comprising administering to the animal a conjugate comprising the cargo linked to a FIBCD1-targeting agent via a linking moiety. In certain embodiments, the administration results in delivery of the cargo to the cell by FIBCD1-mediated endocytosis of the conjugate. In certain embodiments, the cargo enters the cell via endocytosis and further exits the cell via exocytosis (i.e., is capable of transcytosis). In certain embodiments, the cargo is a chemical or biological agent described herein. In certain embodiments, the cargo is a therapeutic agent. The term “FIBCD1-targeting agent” described herein refers to an agent having binding affinity for human FIBCD1. In certain embodiments, the FIBCD1-targeting agent is a compound described herein. In certain embodiments, the conjugate is a conjugate described herein. In certain embodiments, the conjugate is administered intravenously, or orally.

### Administration

Macrocyclic compounds, or conjugates described herein can be formulated as pharmaceutical compositions and administered to a mammalian host, such as a human patient in a variety of forms adapted to the chosen route of administration, i.e., orally or parenterally, by intravenous, intramuscular, intraperitoneal, intrathecal, topical, nasal, inhalation, pulmonary, suppository, sub dermal osmotic pump, intradermal or subcutaneous routes.

Thus, compounds may be systemically administered, e.g., orally or parenterally, in combination with a pharmaceutically acceptable vehicle such as an inert diluent or an assimilable edible carrier. They may be provided as a lyophilized formulation (e.g., with trehalose or sucrose as cryo-lyoprotectant, and/or mannitol as bulking agent), or enclosed in hard or soft shell gelatin capsules, may be compressed into tablets. For oral therapeutic administration, the active therapeutic compound may be combined with one or more excipients and used in the form of ingestible tablets, buccal tablets, troches, capsules, elixirs, suspensions, syrups, wafers, and the like. Such compositions and preparations should contain at least 0.1% of active therapeutic compound. The percentage of the compositions and preparations may, of course, be varied and may conveniently be between about 2 to about 60% of the weight of a given unit dosage form. The amount of active therapeutic compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The compound may also be administered subcutaneously, intradermally, intranasally, intramuscularly, intrathecally, intravenously or intraperitoneally by infusion or injection. A compound may also be administered via intranasal and/or pulmonary delivery (e.g., delivered as a spray or mist). Additionally, the compound be administered by local injection, such as by intrathecal injection, epidural injection or peri-neural injection using a scope. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds described herein to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compound of the invention can be determined by comparing their in vitro activity, and in vivo activity in animal models. Methods for the extrapolation of effective dosages in mice, and other animals, to humans are known to the art; for example, see U.S. Pat. No. 4,938,949.

The amount of the therapeutic compound required for use in treatment will vary with the route of administration, the nature of the condition being treated and the age and condition of the patient and will be ultimately at the discretion of the attendant physician or clinician.

The compound may be conveniently formulated in unit dosage form. In one embodiment, the invention provides a composition comprising a compound formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Macrocyclic compounds as described herein, peptidyl residue thereof, or conjugates thereof, can also be administered in combination with other therapeutic agents, for example, an anti-cancer agent or an agent for treating diabetes. Accordingly, in one embodiment the invention also provides a composition comprising a macrocyclic compound as described herein, peptidyl residue thereof, or a conjugate thereof, at least one other therapeutic agent, and a pharmaceutically acceptable diluent or carrier. The invention also provides a kit comprising a macrocyclic compound as described herein, peptidyl residue thereof, or a conjugate thereof, at least one other therapeutic agent, packaging material, and instructions for administering the macrocyclic compound as described herein, peptidyl residue thereof, or a conjugate thereof, and the other therapeutic agent or agents to a mammal to treat a disease or condition (e.g., cancer or diabetes).

### Certain Embodiments

Embodiment 1. A macrocyclic compound comprising a pentapeptide segment X₁-X₂-X₃-X₄-X₅, wherein:
X₁, X₂, X₃, X₄, and X₅ are each independently a residue of Phe, Tyr, Thr, Ser, or homo-Ser;
wherein Phe or Tyr is optionally substituted with one or more halo or hydroxy group on the phenyl ring; and
wherein Thr, Ser, or homo-Ser is optionally substituted with one or more halo, hydroxy, (C₁-C₆)alkyl, (C₃-C₆)cycloalkyl, aryl, or heteroaryl, wherein the (C₁-C₆)alkyl, (C₃-C₆)cycloalkyl, aryl, or heteroaryl is optionally substituted with one or more halo or hydroxy group;
or a peptidyl residue or a salt thereof,
provided the pentapeptide segment is not Tyr-Tyr-Thr-Tyr-Thr (SEQ ID NO:1).

Embodiment 2. The compound of embodiment 1, comprising a peptide segment having 5 to 9 amino acids in length, or a peptidyl residue thereof, or a salt thereof.

Embodiment 3. The compound of any one of embodiments 1-2, wherein the pentapeptide segment X₁-X₂-X₃-X₄-X₅ comprises five hydroxy groups.

Embodiment 4. The compound of any one of embodiments 1-3, wherein X₁ is a residue of Phe or Tyr.

Embodiment 5. The compound of any one of embodiments 1-4, wherein X₂ is a residue of Thr, Ser, or homo-Ser.

Embodiment 6. The compound of any one of embodiments 1-5, wherein X₃ is a residue of Phe or Tyr.

Embodiment 7. The compound of any one of embodiments 1-5, wherein X₃ is a residue of Thr, Ser, or homo-Ser.

Embodiment 8. The compound of any one of embodiments 1-6, wherein X₄ is a residue of Phe or Tyr.

Embodiment 9. The compound of any one of embodiments 1-8, wherein X₅ is a residue of Thr, Ser, or homo-Ser.

Embodiment 10. The compound of any one of embodiments 1-9, wherein X₁ and X₄ are each independently a residue of Phe or Tyr.

Embodiment 11. The compound of any one of embodiments 1-10, wherein X₁, X₃ and X₄ are each independently a residue of Phe or Tyr.

Embodiment 12. The compound of any one of embodiments 1-11, wherein X₂ and X₅ are each independently a residue of Thr, Ser, or homo-Ser.

Embodiment 13. The compound of any one of embodiments 1-12, wherein X₁ and X₄ are each independently a residue of Phe or Tyr; and X₂ and X₅ are each independently a residue of Thr, Ser, or homo-Ser.

Embodiment 14. The compound of any one of embodiments 1-13, wherein the pentapeptide segment X₁-X₂-X₃-X₄-X₅ is selected from the group consisting of

(SEQ ID NO: 2)
Thr-Tyr-Tyr-Thr-Tyr,
(SEQ ID NO: 3)
Tyr-Thr-Tyr-Tyr-Thr,
(SEQ ID NO: 4)
Tyr-Tyr-Tyr-Tyr-Tyr,
(SEQ ID NO: 6)
Tyr-Ser-Tyr-Tyr-Ser,
and
(SEQ ID NO:7)
Ser-Tyr-Tyr-Ser-Tyr.

Embodiment 15. The compound of any one of embodiments 1-14, from N terminal to C terminal, having formula I:

(Rₙ)₂N-c[X₀-X₁-X₂-X₃-X₄-X₅-X₆]—C(═O)—R_{c} (I)

wherein:

X₀ is a residue of an amino acid,

X₆ is a residue of an amino acid,

each Rₙ is independently H, (C₁-C₆)alkyl, or (C₁-C₆)alkanoyl, and

R_{c} is hydroxy or —N(R_{f})₂, wherein each R_{f} is independently H or (C₁-C₆)alkyl.

Embodiment 16. The compound of claim 15, wherein Rₙ are each H.

Embodiment 17. The compound of any one of embodiments 15-16, wherein X₀ is a residue of Pra.

Embodiment 18. The compound of any one of embodiments 15-17, wherein X₆ is a residue of Az4.

Embodiment 19. The compound of any one of embodiments 15-18, comprising a cyclic heptapeptide sequence selected from the group consisting of

c[Pra-Thr-Tyr-Tyr-Thr-Tyr-Az4] (SEQ ID NO:9),

c[Pra-Tyr-Thr-Tyr-Tyr-Thr-Az4] (SEQ ID NO:10),

c[Pra-Tyr-Tyr-Tyr-Tyr-Tyr-Az4] (SEQ ID NO:11),

c[Pra-Tyr-Ser-Tyr-Tyr-Ser-Az4] (SEQ ID NO:13), and

c[Pra-Ser-Tyr-Tyr-Ser-Tyr-Az4] (SEQ ID NO:14).

Embodiment 20. The compound of any one of embodiments 1-19, having formula II:

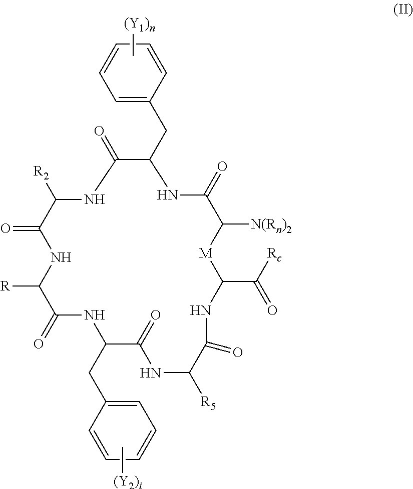

wherein
R₂ and R₅ are each independently (C₁-C₃)alkyl substituted with one or more hydroxy groups,
R is benzyl optionally substituted with one or more halo or hydroxy groups on the phenyl ring, or (C₁-C₃)alkyl substituted with one or more hydroxy groups,
each Y₁ is independently halo or hydroxy,
each Y₂ is independently halo or hydroxy,
h and i are each independently 0, 1, 2, 3, 4, or 5,
each Rₙ is independently H, (C₁-C₆)alkyl, or (C₁-C₆)alkanoyl,
R_{c} is hydroxy or —N(R_{f})₂,
each R_{f} is independently H or (C₁-C₆)alkyl,
M is divalent, branched or unbranched, saturated or unsaturated, hydrocarbon chain having from 2 to 16 carbon atoms, wherein one or more of the carbon atoms is optionally replaced by (—O—), (—S—), (—NRₐ—), (C₃-C₈)cycloalkyl, aryl, or heteroaryl, wherein the chain is optionally substituted on carbon with one or more substituents selected from the group consisting of halo, hydroxy, mercapto, oxo (═O), and thioxo(═S); wherein Rₐ is a H or (C₁-C₆)alkyl.

Embodiment 21. The compound of claim 20, having formula II(a):

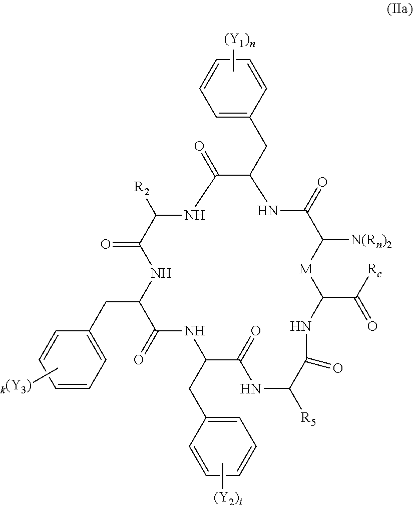

wherein each Y₃ is independently halo or hydroxy, and
   k is 0, 1, 2, 3, 4, or 5.

Embodiment 22. The compound of claim 20, having formula II(b):

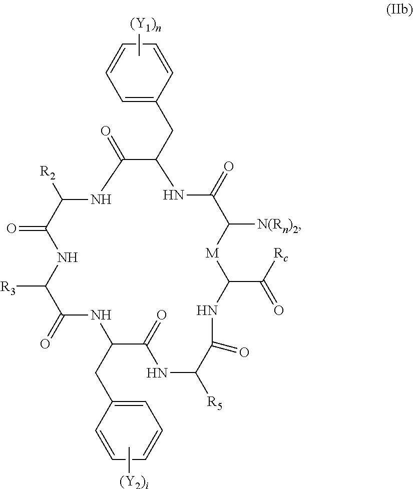

R₃ is (C₁-C₃)alkyl substituted with one or more hydroxy groups.

Embodiment 23. The compound of claim 22, wherein R₂, R₃, and R₅ are each independently hydroxymethyl, 1-hydroxyethyl, or 2-hydroxyethyl.

Embodiment 24. The compound of any one of embodiments 1-23, comprising a cyclic heptapeptide sequence of

(SEQ ID NO: 10)
c[Pra-Tyr-Thr-Tyr-Tyr-Thr-Az4]
or
(SEQ ID NO: 13)
c[Pra-Tyr-Ser-Tyr-Tyr-Ser-Az4].

Embodiment 25. The compound of any one of embodiments 1-24, having the structure of

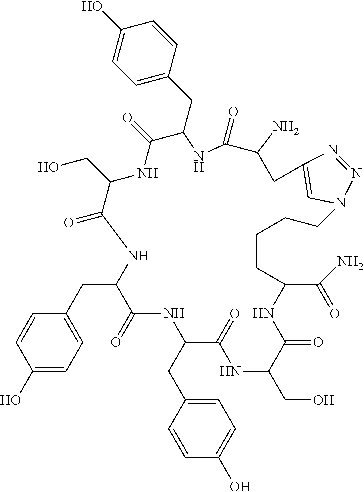 [SMILES: NC(=O)C1CCCCn2cc(nn2)CC(N)C(=O)NC(Cc2ccc(O)cc2)C(=O)NC(CO)C(=O)NC(Cc2ccc(O)cc2)C(=O)NC(Cc2ccc(O)cc2)C(=O)NC(CO)C(=O)N1]

Embodiment 26. The compound of any one of embodiments 1-25, wherein the compound is capable of entering a [symbol] [symbol] cell via endocytosis.

Embodiment 27. The compound of any one of embodiments 1-26, wherein the compound is [symbol] capable of entering a [symbol] cell via caveolin and/or dynamin-dependent endocytosis.

Embodiment 28. The compound of any one of embodiments 1-27, wherein the compound is capable of binding human fibrinogen C domain-containing protein 1 (FIBCD1).

Embodiment 29. A conjugate having the structure of formula (III):

P-L-cargo (III)

wherein:
P is a peptidyl residue of the compound according to any one of embodiments 1-28;
cargo is a chemical agent or biological agent (e.g., a detectable agent, synthetic biodegradable polymer, peptide, polypeptide, or polynucleotide);
L is a linking moiety having the structure:

—W—Z—T—
W is selected from the group consisting of a bond, —O—, —S—, —C(═O)—, —N(R_{S})—, —C (═O)NH—, —C(═S)NH—, —C(═O)O—, —C(═O)S—, —NHSO₂—, —OC(═O)NH—, —NHC(═O)NH—, and —NHC(═S)NH—, wherein R_{S} is H or (C₁-C₆)alkyl;
T is selected from the group consisting of a bond, —O—, —S—, —C(═O)—, —C (═O)NH—, —C(═S)NH—, —C(═O)O—, —C(═O)S—, —NHSO₂—, —OC(═)NH—, —NHC(═O) NH—, and —NHC(═S)NH—, wherein Rᵤ is H or (C₁-C₆)alkyl;
Z is selected from the group consisting of a bond, or a divalent, branched or unbranched, saturated or unsaturated, hydrocarbon chain having from 2 to 16 carbon atoms, wherein one or more of the carbon atoms is optionally replaced by (—O—), (—S—), (—NRₚ—), (C₃-C₈)cycloalkyl, aryl, or heteroaryl, wherein Rₚ is H or (C₁-C₆)alkyl, wherein the hydrocarbon chain is optionally substituted on carbon with one or more substituents selected from the group consisting of halo, hydroxy, amino, mercapto, oxo(═O), and thioxo(═S).

Embodiment 30. The conjugate of claim 29, wherein the cargo is a small molecule compound having molecular weight smaller than 1,000 g/mol.

Embodiment 31. The conjugate of any one of embodiments 29-30, wherein the N terminus of the peptide is conjugated to the cargo via an amide bond.

Embodiment 32. The conjugate of any one of embodiments 29-31, wherein the C terminus of the peptide is conjugated to the cargo via an amide or ester bond.

Embodiment 33. The conjugate of any one of embodiments 29-32, wherein the cargo is a fluorescent dye, a chemotherapeutic agent, or a protein.

Embodiment 34. The conjugate of any one of embodiments 29-33, wherein the cargo is Rhodamine B, Alexa Fluor 555 or Alexa Fluor 647, gemcitabine, or insulin.

Embodiment 35. A method for intracellular delivery, comprising contacting a cell with the compound according to any one of embodiments 1-28 or the conjugate of any one of embodiments 29-34.

Embodiment 36. The method of claim 35, wherein the compound or the conjugate is delivered to an endosome, lysosome, and/or cytosol of the cell.

Embodiment 37. The method of claim 36, wherein the compound or the conjugate enters the cell via

endocytosis and exits the cell via exocytosis.

Embodiment 38. The method according to any one of embodiments 35-37, wherein the cell is a ciliated epithelial cell.

Embodiment 39. A method for treating a disease or condition, comprising administering the conjugate of any one of embodiments 29-34 to a subject in need thereof, wherein the cargo is a therapeutic agent.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, (C₁-C₆)alkyl, (C₂-C₆)alkyl, (C₁-C₃)alkyl, and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and higher homologs and isomers.

The term “alkenyl” refers to an unsaturated alkyl radical having one or more double bonds. Examples of such unsaturated alkyl groups include vinyl, 2-propenyl, crotyl, 2-isopentenyl, 2-(butadienyl), 2,4-pentadienyl, 3-(1,4-pentadienyl) and the higher homologs and isomers.

The term “alkynyl” refers to an unsaturated alkyl radical having one or more triple bonds. Examples of such unsaturated alkyl groups ethynyl, 1- and 3-propynyl, 3-butynyl, and higher homologs and isomers.

The term “cycloalkyl” or “carbocycle” refers to a saturated or partially unsaturated (non-aromatic) all carbon ring having 3 to 8 carbon atoms (i.e., (C₃-C₈) cycloalkyl). The term also includes multiple condensed, saturated all carbon ring systems (e.g., ring systems comprising 2, 3 or 4 cycloalkyl rings). Accordingly, cycloalkyl includes multicyclic cycloalkyls such as a bicyclic cycloalkyls (e.g., bicyclic cycloalkyls having about 3 to 15 carbon atoms, about 6 to 15 carbon atoms, or 6 to 12 carbon atoms such as bicyclo[3.1.0]hexane and bicyclo[2.1.1]hexane), and polycyclic cycloalkyls (e.g tricyclic and tetracyclic cycloalkyls with up to about 20 carbon atoms). The rings of the multiple condensed ring system can be connected to each other via fused, spiro and bridged bonds when allowed by valency requirements. For example, multicyclic cycloalkyls can be connected to each other via a single carbon atom to form a spiro connection (e.g., spiropentane, spiro[4,5]decane, etc), via two adjacent carbon atoms to form a fused connection (e.g., cycloalkyls such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a cycloalkyl portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl.

The term “halo” or “halogen” refers to bromo, chloro, fluoro or iodo. In some embodiments, halogen refers to chloro or fluoro.

As used herein, the term “Linking moiety”, “linker”, or the variable “L” refers to a divalent functional group that covalently bonds two or more moieties in a conjugate or material. For example, the linking moiety can serve to covalently bond a targeting and/or cell penetrating compound to a cargo compound (e.g., a small molecule compound or a polymer). Useful bonds for connecting linking moieties to a compound and other materials include, but are not limited to, amide, thioamide, amine, ester, thioester, ether, thioether, sulfonamide, carbamate, carbamide, thiourea, or siloxane bond. In certain embodiments, the linking moiety comprises an alkyl chain of C₁C₁₂, wherein one or more carbon is optionally replaced with —O—, —N(Rₚ)—, —S—, wherein Rₚ is H or (C₁-C₆) alkyl.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that certain compounds described herein have a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

When a bond in a compound formula herein is drawn in a non-stereochemical manner (e.g. flat), the atom to which the bond is attached includes all stereochemical possibilities.

The term “subject,” “individual,” and “patient,” as used interchangeably herein, refer to a mammal, including but not limited to humans, higher non-human primates, rodents, cows, horses, pigs, sheep, dogs, and cats. In one embodiment, the subject is a human.

The term “therapeutically effective amount,” in reference to treating a disease/condition, refers to an amount of a therapeutic agent alone (e.g., a conjugate described herein) or as contained in a pharmaceutical composition that is capable of having any detectable, positive effect on any symptom, aspect, or characteristics of a disease state/condition when administered as a single dose or in multiple doses. Such effect need not be absolute to be beneficial.

The terms “treat” and “treatment” refer to both therapeutic treatment and prophylactic or preventative measures, wherein the object is to prevent or decrease an undesired physiological change or disorder, such as a cancer or diabetes. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

### Example 1 Endocytosis Promoting Cyclic Peptides

Described in this Example is a group of hydrophilic endocytosis-promoting peptides (EPPs) rich in hydroxyl groups and have no positive charge. These EPPs can transport a wide array of small-molecule cargos into a diverse panel of animal cells. Without wanting to be bound by theory, mechanistic studies reveal that the EPPs may enter the cells through a caveolin- and dynamin-dependent endocytosis pathway, mediated by the surface receptor fibrinogen C domain-containing protein 1 (FIBCD1). After endocytosis, EPPs traffic through early and late endosomes within 30 minutes, and partition among the cytosol, the late endosomes and lysosomes. The EPPs also demonstrated prominent transcytosis abilities. This Example shows that positive charge is not an indispensable feature for hydrophilic cell penetrating peptides, and provides a new category of molecularly well-defined delivery tags for chemical biology and drug delivery applications.

### Introduction

Transporting cargo molecules into the cell is a critical and everlasting goal for many medicinal chemistry and chemical biology studies. ^{1,2} As the natural route to bring molecules into the cell, the endocytosis process has been a primary focus in the field, especially when hydrophilic cargo molecules are involved.³⁻⁵ A broad spectrum of endocytosis-promoting modalities has been discovered and developed.^{6,7} On one hand, various nanoparticles, such as liposomes and micelles, can serve as delivery vehicles and bring native-state cargo molecules across the cell membrane.^{8,9} Some nanoparticle delivery platforms have already demonstrated clinical success.¹⁰ On the other hand, smaller endocytosis-promoting moieties can be conjugated to cargo molecules and carry them into the cells.^{11,12} Compared with nanoparticle-based platforms, molecularly well-defined delivery tags allow for a more straightforward manufacturing process and a clearer path for medicinal chemistry optimizations. Representative examples of these molecularly well-defined groups include folate, transferrin, miniature proteins, and, notably, cell-penetrating peptides (CPPs).^{13,17}

Over the past few decades, a very diverse panel of CPPs has been developed.¹⁸ Early examples such as the transactivator of transcription (TAT) peptide and RGD sequence have proven capable of delivering various cargo molecules, and they continue to be widely employed to date.^{19,20} More recently, advanced sequences, such as penetratin, iRGD, and CPP12, have demonstrated superior delivery efficacy.²¹⁻²³ Currently, improving the cell-penetrating ability, intracellular targeting, and biocompatibility of these CPPs remains a very active and attractive research field.²⁴⁻²⁸

Despite the prominent sequence variations, these previously studied CPPs share a common feature—they are positively charged.²⁹ This positive charge promotes the initial interaction with the extracellular matrix and the cell membrane, which is the prerequisite of endocytosis.³⁰ Consequently, the current dogma regards this positive charge as an indispensable component of CPPs.^{17,31,32} Nevertheless, considering that even heavily negatively charged nanoparticles can still be taken up by the cells efficiently,³³ one may question the necessity of relying on positive charges to induce endocytosis. Herein, presented in this Example is a series of endocytosis-promoting peptides (EPPs) that do not have positive charges. These cyclic peptides are rich in hydroxyl groups and can bring various cargo molecules into a diverse collection of cells.

### Results

Hydroxyl-Rich Cyclic Peptides Bring Cargo Molecules into Cells

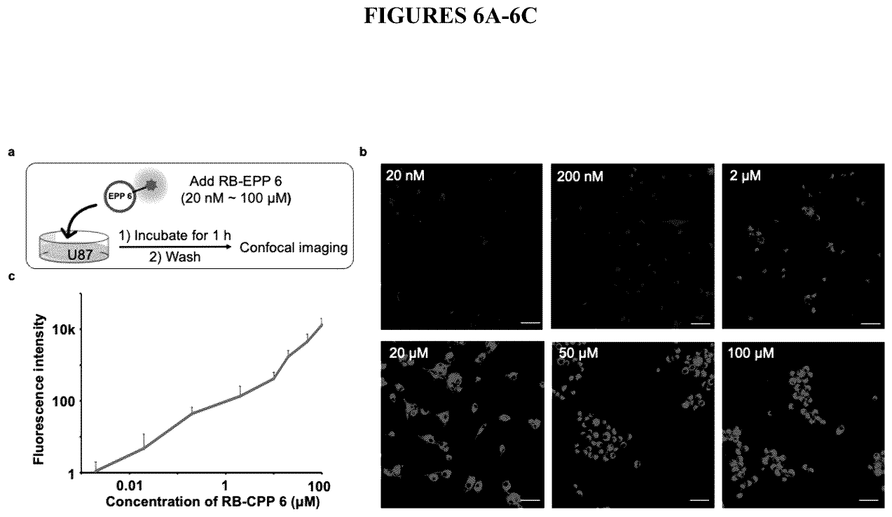

Seven hydroxyl-rich cyclic peptides using tyrosine, threonine, and serine as the building blocks were prepared. These peptides had five modular amino acid positions and were cyclized through a triazole ring (FIG. 1a, b), and the exposed N terminal allowed for cargo conjugation. In one embodiment, EPPs were linked to a Rhodamine B (RB) tag and their abilities to enter the cells were assessed. U87 cell line was chosen as the model system and confocal imaging was used to evaluate the intracellular fluorescence (FIG. 1c). As shown in FIG. 1d, these EPPs had differential abilities to enter the cells, with EPP6 demonstrating the highest efficiency. Compared with the benchmark, TAT peptide, EPP6 led to ˜2× fluorescence intensity in the cells. More interestingly, it was found that increasing EPP6 concentration led to higher fluorescence signals across a wide range (FIG. 6). Subsequent studies of this Example focus on EPP6 as the model compound.

Considering that the size of EPP6 and the RB tag was comparable, and RB was known to interact with the cell membrane, it needs to be confirmed that EPP6, rather than the RB tag, was the critical component that led to the uptake. Therefore, EPP6 was conjugated to different fluorescent dye molecules and similar uptake experiments were performed using U87 cells. The results proved that EPP6 could bring all these cargo molecules into U87 cells (FIG. 1e). It is worth pointing out that AF555 and AF647 are negatively charged dyes and exhibit negligible interaction with the cell membrane. Therefore, the results confirmed that it was the EPP6 moiety that enabled the uptake.

It was then tested if the observed EPP6 uptake was limited to U87 cells. RB-EPP6 was incubated with different cell lines and the intracellular fluorescence intensities were evaluated. As shown in FIG. 1f, EPP6 was able to enter a wide array of human cancer and noncancer cells, as well as non-human cells. Nevertheless, the resulting intracellular fluorescence intensities varied significantly across different cell lines, which prompted further investigation on the mechanism of cell entry.

### EPP6 Enters the Cells Through Caveolin- and Dynamin-Dependent Endocytosis

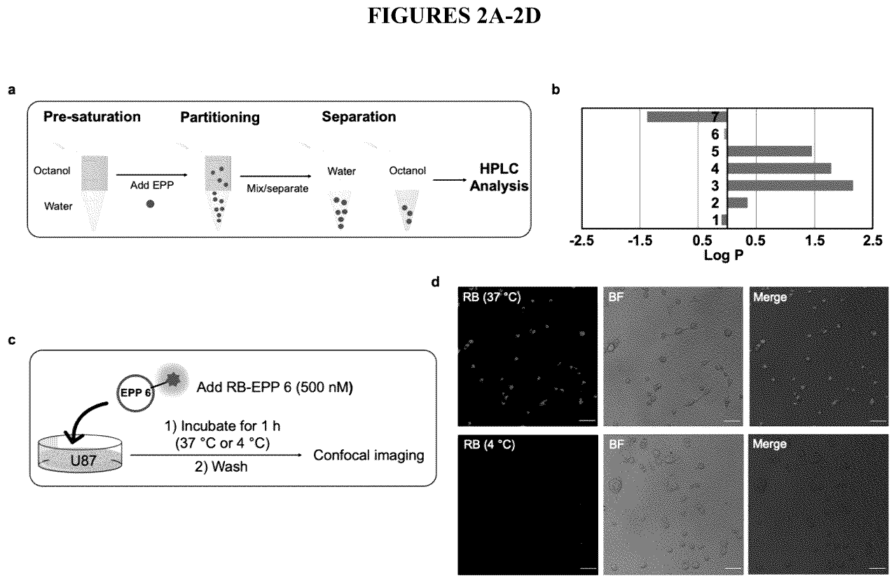

Unlike the well-studied positively charged CPPs (TAT, penetrating, poly-R, CPP12, etc.), these EPPs did not have charged residues. On the other hand, the hydroxyl groups may form intramolecular hydrogen bonds that render the EPPs hydrophobic. Because hydrophobic peptides are known to penetrate the cell membrane autonomously, it was possible that the EPPs entered the cells due to hydrophobicity. To test this hypothesis, the octanol partitioning experiment was performed to assess the hydrophobicity of RB-EPPs (FIG. 2a). Interestingly, most of the RB-EPPs were not strongly hydrophobic (FIG. 2b). Particularly, the best performing candidate, RB-EPP6, appeared to be hydrophilic, while the worst one, RB-EPP3, was the most hydrophobic. These results proved that hydrophobicity was not the driving force of EPP function.

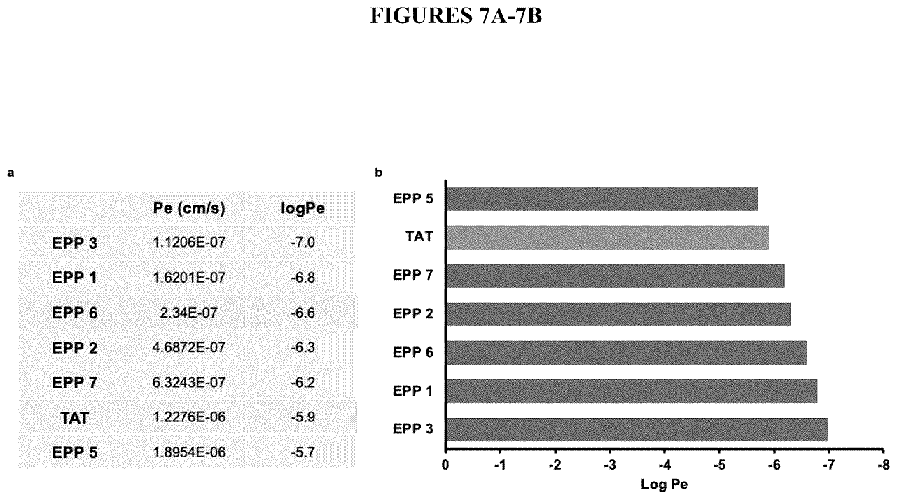

It was then tested whether EPPs entered the cells through passive diffusion or active transportation. U87 cells was incubated with RB-EPP6 at 37° C. and 4° C., and subsequently confocal imaging was used to assess the intracellular fluorescence intensities (FIG. 2c). As shown in FIG. 2d, the low-temperature treatment led to no fluorescence signal in the cells, suggesting that RB-EPP6 entered the cells through an energy-dependent process. To validate these findings, the parallel artificial membrane permeability assay (PAMPA) was further performed to evaluate the passive diffusion abilities of the EPPs (FIG. 7). It was found that EPPs could not pass through the artificial membrane, which further supported the conclusion that EPPs entered the cells through active transportation mechanisms.

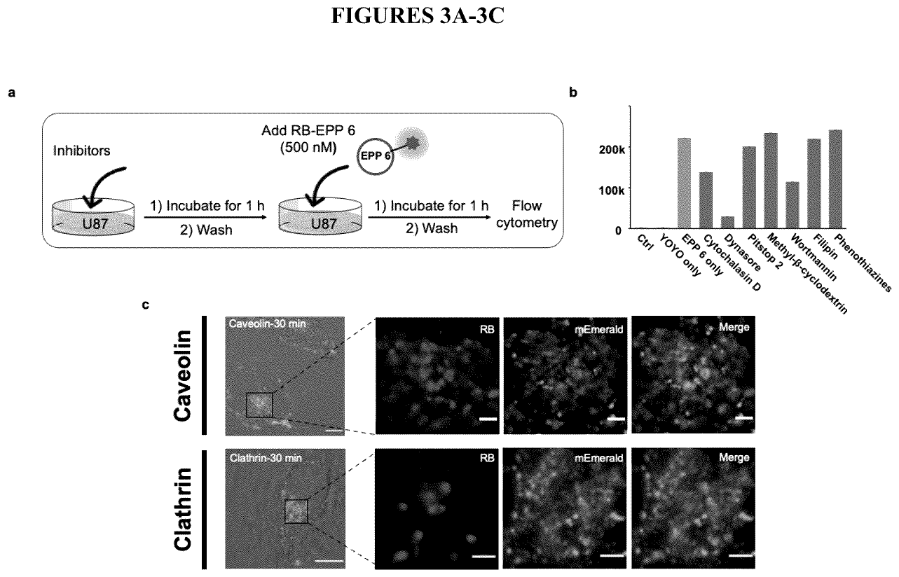

Considering the size of the EPPs, and that different dye conjugates were all able to enter the cells, the transportation was unlikely to involve transmembrane transporter proteins or ion channels. Therefore, an endocytosis pathway might be the more plausible mechanism. To identify the critical components in EPP transportation, a panel of inhibitors targeting various parts of common endocytosis pathways was used. U87 cells were incubated with RB-EPP6 in the presence of these inhibitors and flow cytometry was used to compare the resulted fluorescence intensities (FIG. 3a). As shown in FIG. 3b, dynamin inhibition (Dynasore) almost completely suppressed RB-EPP6 uptake, which suggested a dynamin-dependent endocytosis pathway. Similarly, inhibition of actin polymerization (cytochalasin D) and PI3K signaling (wortmannin) caused significantly decreased RB-EPP6 uptake, albeit not as prominent as that from the dynamin inhibition. In contrast, inhibitors of clathrin-mediated endocytosis (Pitstop 2 and phenothazine) did not affect the uptake, which pointed to a clathrin-independent process. Based on the current picture of endocytosis pathways, these results hinted at a caveolin-mediated endocytosis pathway.

To further delineate the roles of caveolin and clathrin, U87 cells was transfected with mEmerald-caveolin and mEmerald-clathrin plasmids, the cells were incubated with RB-EPP6, and the resulted intracellular fluorescence were assessed. It was observed that the RB-EPP6 signal significantly overlapped with the mEmerald-caveolin fluorescence, while no overlap existed between RB-EPP6 and mEmerald-clathrin (FIG. 3c). This result further validated the hypothesis that EPP6 enters the cells through the caveolin-dependent pathway.

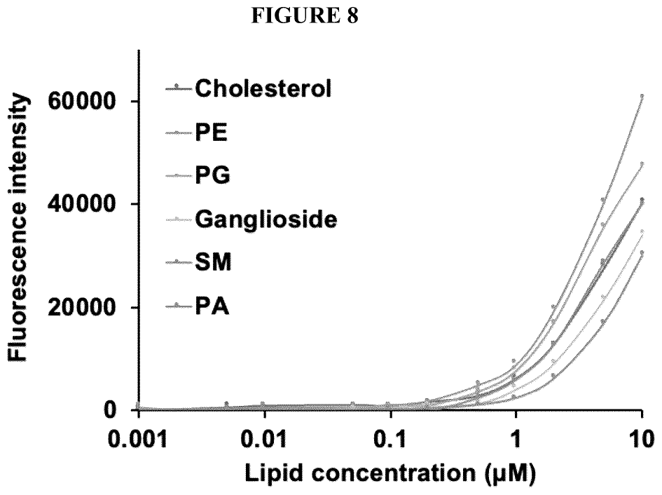

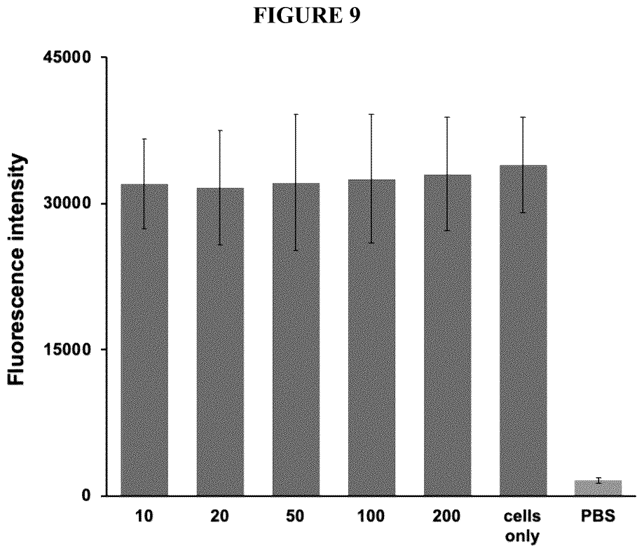

Interestingly, it was further found that cholesterol extraction (methyl-β-cyclodextrin) and cholesterol binding (filipin) did not inhibit RB-EPP6 uptake (FIG. 3b). These results were unusual because most caveolin-mediated endocytosis mechanisms involve cholesterol and lipid rafts. To validate this finding, the binding affinity between RB-EPP6 and cell membrane lipid components was tested. The results confirmed that there was no appreciable interaction between RB-EPP6 and lipids (FIG. 8). Another popular pathway of caveolin-dependent endocytosis relies on heparan sulfate proteoglycans (HSPG), which is implicated in the uptake of some well-established CPPs and many other macromolecules. However, high concentrations of heparin did not affect RB-EPP6 uptake (FIG. 9) which suggested that HSPG was not involved. Taken together, these results suggested that EPP6 took an uncommon mechanism within the premises of dynamin- and caveolin-dependent endocytosis pathway, perhaps through a receptor-mediated endocytosis process.

### FIBCD1 is a Surface Receptor for EPP6 Recognition

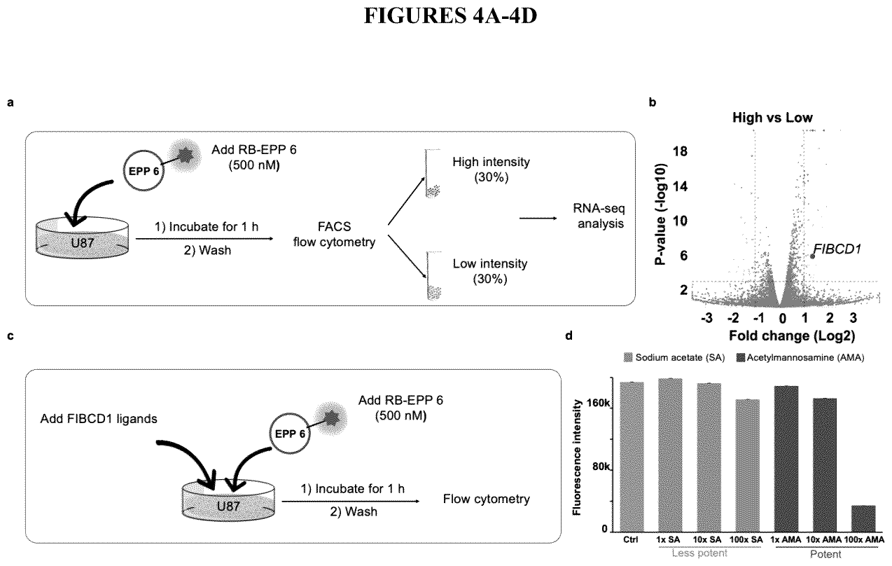

To better understand the EPP6 endocytosis mechanism, further study was conducted to identify the genes involved in the uptake. U87 cells was incubated with RB-EPP6 and a fluorescence-activated cell sorter was used to isolate the top 30% and the bottom 30% populations based on the RB-channel cellular fluorescence. RNAs from these two populations as well as the control (untreated) cells were extracted and transcriptome analysis by RNA-seq was carried out (FIG. 4a). Three biological repeats were prepared for each condition, and on average 30 million reads were generated per sample. The raw data was processed using fastp, STAR, and featureCounts to generate annotated gene lists, and the differentially expressed genes between the samples were identified by DESeq2. Here, the aim was to find upregulated genes in the high-uptake samples that could be receptors for EPP6 uptake. The high-uptake samples were compared with the low-uptake ones. Based on the criteria of p<0.0001 and >2-fold changes, one gene was found, fibrinogen C domain-containing protein 1 (FIBCD1), stood out during the process and it was hypothesized that it was the receptor responsible for EPP6 uptake (FIG. 4b).

FIBCD1 is a type II transmembrane receptor, and it is known to induce endocytosis. Its natural ligands are mono- and oligosaccharides, including acetylmannosamine, chitin, β-1,3-glucan, and galactomannan. Notably, significant structural similarities exist between these ligands and the EPPs, as they all present abundant hydroxyl groups. Such similarities supported the hypothesis that FIBCD1 was the receptor for EPP6 uptake. To further validate the hypothesis, a competition experiment was performed where U87 cells were incubated with RB-EPP6 with and without acetylmannosamine—a potent FIBCD1 ligand (FIG. 4c). The results showed that acetylmannosamine was able to inhibit RB-EPP6 uptake in a concentration-dependent manner (FIG. 4d).

### Intracellular Fate of EPP6

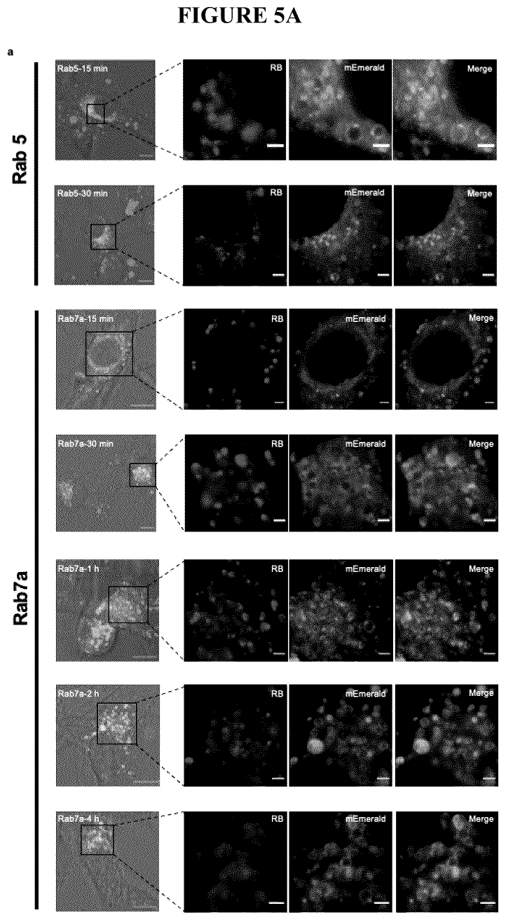

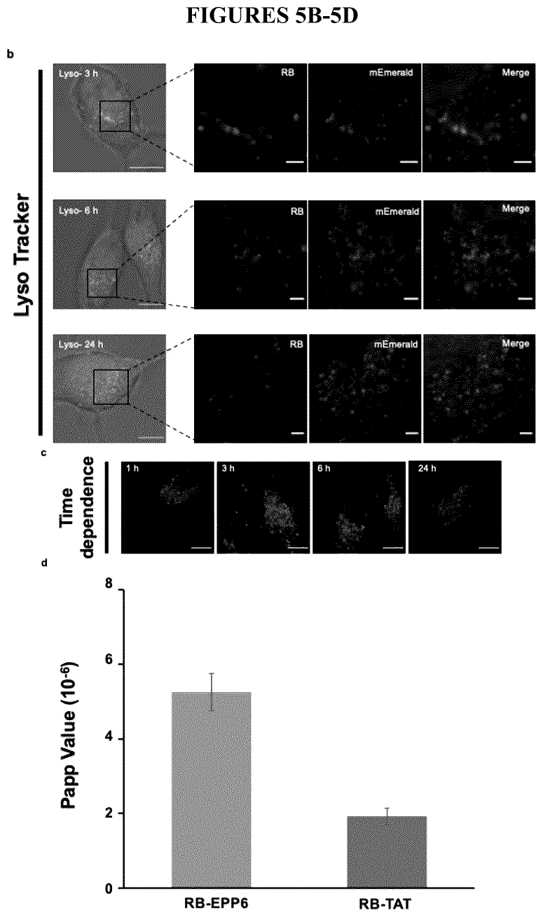

The fate of EPP6 after endocytosis was investigated. Typical cargo molecules go through the early endosome—late endosome—lysosome pathway after endocytosis, while many well-established CPPs could escape from the endosomes and achieve cytosolic delivery. It was hypothesized that EPP6 would adopt a similar pathway. To label the endosomes, U87 cells was transfected with EGFP-Rab5 (early endosome marker) and mEmerald-Rab7a (late endosome marker). These cells were incubated with RB-EPP6 and confocal microscopy was used to analyze the colocalization of RB-EPP6 with the fluorescent markers. As shown in FIG. 5a, after 15-min, RB-EPP6 appeared in distinct Rab5-coated vesicles, indicating that RB-EPP6 traveled into early endosomes quickly after the endocytosis. Meanwhile, no obvious colocalization was observed between Rab7a-labeled vesicles and RB-EPP6. At 30 minutes, RB-EPP6 disappeared from the Rab5-labeled vesicles and appeared in Rab7a-labeled vesicles. This transition suggested that RB-EPP6 trafficked into late endosomes within 30 minutes. Interestingly, it was found that a significant amount of RB-EPP6 remained inside Rab7a+vesicles even after 4 hours.

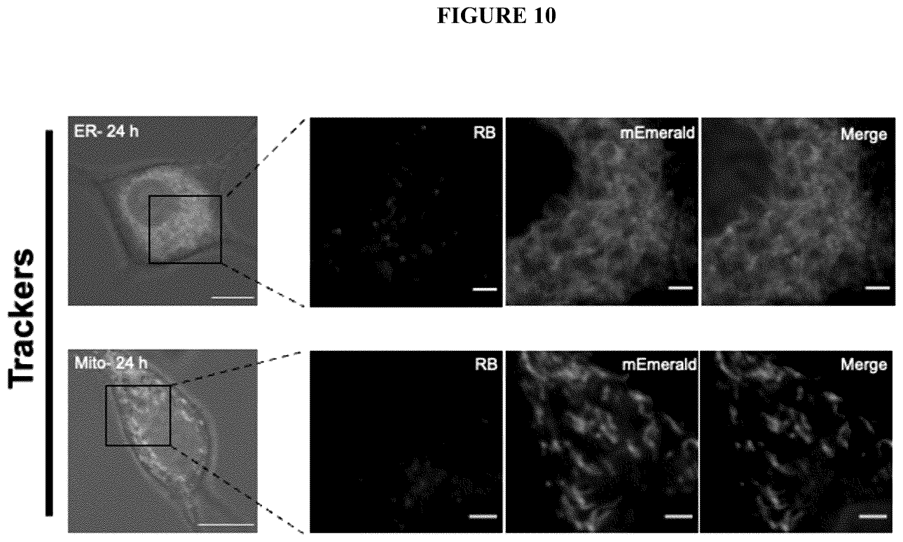

To further investigate the intracellular trafficking of RB-EPP6 after endocytosis, LysoTracker was used to label the lysosomes and the signal colocalization was assessed. It was found that some RB-EPP6 signals appeared in lysosomes after 3 hours (FIG. 5b). Nevertheless, many RB-EPP6-containing vesicles were not in lysosomes even after 6 hours, which was consistent with the results that the RB-EPP6 signal remained in Rab7a+vesicles. It is worth pointing out that in addition to the obvious punctuated RB-EPP6 signal, there was also significant smearing of the signals that indicated cytosolic distribution. Indeed, such smeared signals were obvious at 1 hour and became more prominent after 3 hours (FIG. 5c). Interestingly, some signals remained even after 24 hours. More importantly, a significant amount of the remaining signal was punctuated. Because endosomes and lysosomes do not have a long lifetime, these punctate signals suggested that RB-EPP6 remained in other long-live vesicles. In addition, it was also validated that RB-EPP6 did not traffic into the mitochondria or the endoplasmic reticulum (FIG. 10).

Considering that the intracellular signal decreased over time, it was hypothesized that a part of the cargo molecules might exit the cells through exocytosis, which might further enable transcytosis. To test this hypothesis, tight cell monolayers in a trans-well apparatus was prepared using MDCK cells, and the apical transcytosis rate of RB-EPP6 was assessed. RB-TAT was also included for comparison. After three hours of incubation, the target molecule concentrations in the apical and basal chambers were quantified, and the Papp values were calculated. It was found that RB-EPP6 exhibited a Papp value of 5.25×10⁻⁶, which was more than twice that of RB-TAT (FIG. 5d).

### Discussion

Molecularly defined delivery tags can bring cargo molecules into cells through endocytosis, and they have profound research and therapeutic applications. Because their sizes are often comparable to, if not smaller than, the cargo, the delivery efficiency might be cargo-dependent. Consequently, there are no universally effective delivery tags for all cargos, and there remains a pressing need to expand the collection. The endocytosis-promoting peptides (EPPs) presented here represent a new category of delivery tags. They are hydrophilic and uncharged, and they are able to transport a wide array of small-molecule cargos into a diverse panel of animal cells.

The lack of charge is one key feature that differentiates EPPs from conventional CPPs. All existing hydrophilic CPPs are positive charged, which is deemed necessary for the initial steps of endocytosis. Nevertheless, the premise of this notion is that positively charged CPPs interact with negatively charged membrane components such as lipids and HSPGs, which is only a fraction of the mechanisms that can trigger endocytosis. Therefore, it is hypothesized herein that peptide sequences without positive charges may also trigger endocytosis, perhaps through the receptor-mediated pathways. Indeed, these EPP results support this hypothesis and prove that positive charge is not an indispensable part of peptide-based delivery tags.

In this Example, it was found that EPP6 entered cells through FIBCD1-mediated endocytosis that was likely dependent on caveolin and dynamin. FIBCD1 is implicated in pathogen recognition, where it binds to specific saccharides. It is known to induce endocytosis, but the detailed mechanism remains elusive. The results indicate that FIBCD1-mediated endocytosis may require actin, dynamin, and caveolin, which provided a clearer picture of the endocytic process of FIBCD1. On the other hand, these findings also underscore the complexity of endocytosis pathways, especially the caveolin-dependent endocytosis. Existing studies highlight the critical role of lipid rafts in caveolin-dependent endocytosis, as the membrane enrichment of caveolin relies on these cholesterol-rich regions. However, it was found that cholesterol-binding agents (filipin and methyl-cyclodextrin) did not affect RB-EPP6 uptake, proving that caveolin-dependent endocytosis can also take a lipid raft-independent route. In addition, the results echo the emerging opinion that the specificity of inhibitors shall be carefully considered when studying the endocytosis pathway.

These results have delineated the intracellular fate of EPP. The imaging results of RB-EPP6 and EB-EPP6 proved that a significant amount of EPP6 could escape from the endosomes and enter the cytosol, but the molecular mechanism of this process was unclear. Because EPP6 is not charged or hydrophobic, common CPP escape mechanisms such as osmotic rupture and budding are probably not applicable here. Similarly, the punctate signals after 24 hours suggest that some RB-EPP6 trafficked to long-lived vesicles. The identity of these vesicles and the underlying mechanism of this trafficking call for future studies.

The presented EPPs have immediate therapeutic implications. EPP6 could bring strongly negatively charged molecules (AF555 and AF647) into the cell, indicating its application in DNA/RNA delivery. On the other hand, because the expression of caveolin and FIBCD1 varies significantly across different tissues and organs, it is also possible to leverage this difference and tailor the EPP delivery for specific targets. For instance, the high expression levels of FIBCD1 and caveolin in the digestive tract may allow efficient gastrointestinal drug delivery and absorption. In addition, the Papp value of RB-EPP6 (5.3×10⁻⁶) is higher than the cutoff threshold of CNS availability (3.5×10⁻⁶), which hints at its potential of crossing the blood-brain barrier. Finally, the discovery of these EPPs may lead to future discovery of more tags targeting different endocytic receptors, further diversifying the arsenal of delivery tags for research and therapeutic applications.

### Experimental

### Materials

TentaGel S—NH₂ resin (loading capacity 0.28 mmol/g) was purchased from Rapp Polymere GmbH and Rink amide MBHA resin (loading capacity 0.678 mmol/g) from Aapptec (Louisville, KY). All the Fmoc-protected amino acids were purchased from Anaspec (Fremont, CA) except Fmoc-L-propargylglycine (Pra) and Fmoc- Lys(N₃)—OH (Az4), which were purchased from Chempep (Wellington, FL) and Chem-Impex (Wood Dale, IL), respectively. The coupling reagent 2-(1H-benzotriazol-1-yl)-1,1,3,3-tetramethyluronium hexafluorophosphate (HBTU, 99.6%) was obtained from Chem-Impex (Wood Dale, IL). Diisopropylethylamine (DIEA, 99.5%) was purchased from ACROS (Germany). Phenyl isothiocyanate (PhNCS) and triisopropylsilane (TIPS) were obtained from TCI (Portland, OR). Piperidine was purchased from Alfa Aesar (Ward Hill, MA). 5(6)-carboxyfluorescein (Fluo) were obtained from ACROS (Pittsburg, PA). Rhodamine B (RB), cuprous iodide (CuI), and α-cyano-4-hydroxycinnamic acid (CHCA) were obtained from Sigma-Aldrich (St. Louis, MO). N, N′-dimethylformamide (DMF), and dichloromethane (DCM) were purchased from Thermo Fisher Scientific (Waltham, MA). Heparin sodium salt, porcine was purchased from MP Biomedicals (China). Cytochalasin D and Hydroxy Dynasore was bought from Tocris Bioscience (Bristol, United Kingdom). Phenothiazine was purchased from TCI (Portland, OR). Wortmannin was obtained from APExBIO Technology (Boston, MA). Pitstop 2 and filipin III was obtained from Sigma-Aldrich (St. Louis, MO). Methyl-beta-cyclodextrin was bought from Alfa Aesar (Haverhill, MA). Alexa Fluor 555 NHS ester and Alexa Fluor 647 NHS ester were purchased from Life Technologies (Eugene, OR). EGFP-Rab5, mEmerald-Caveolin-C-10, mEmerald-Clathrin-15 and mEmerald-Rab7a-7 were purchased from Addgene (Watertown, MA). The Caveolin-1 CRISPR/Cas9 KO plasmid, FIBCD1 CRISPR/Cas9 KO plasmid, control CRISPR plasmid were purchased from Santa Cruz Biotechnology (Santa Cruz, CA). ER-Tracker, Mito-Tracker and Lyso Tracker and all the antibodies were purchased from Cell Signaling Technology (Danvers, MA).

Preparative Reversed-Phase (RP) High-Performance Liquid Chromatography (HPLC). Preparative HPLC was performed on a Thermo Ultimate 3000BX HPLC instrument, using a Phenomenex C18 reversed-phase preparative column (Kinetex 5 μm EVO, 250×21.2 mm²). Nonlinear gradients of 0-100% acetonitrile (with 0.1% TFA) in water (with 0.1% TFA) were employed, and the gradient parameters were adjusted for each product to achieve desired separation efficiencies. A multiwavelength UV-vis detector was used to monitor the absorbance at 215, 280, 480, and 569 nm.

Analytical HPLC. The purity of the peptide was analyzed on a Thermo Ultimate 3000SD HPLC instrument, using a Phenomenex C18 reversed-phase analytical column (Kinetex 2.6 μm EVO, 250×4.6 mm²). A gradient of 0-100% acetonitrile (with 0.1% TFA) in water (with 0.1% TFA) was employed with a flow rate of 1 mL/min. A UV-vis detector was used to monitor the absorbance at 280 or 560 nm. The purity of all cyclic peptides used for binding assays and biological activity assays was >95%.

Mass Spectrometry. The MS and MS/MS spectra were obtained using a SCIEX 5800 matrix-assisted laser desorption ionization time-of-flight (MALDI-TOF) mass spectrometer.

Solid-Phase Peptide Synthesis. The peptides were synthesized following the standard Fmoc SPPS coupling process. Unless otherwise noted, Rink Amide MBHA resin was used for the synthesis. To couple amino acids to the resin, the Fmoc group on the resin was first removed by 20% piperidine/DMF solution (10 min, three times). Fmoc-AA-OH (3 equiv), DIEA (5 equiv), and HBTU (2.8 equiv) were mixed in DMF for 10 min, and the solution was then introduced to the deprotected resin. The mixture was gently agitated at room temperature for 1 h, followed by draining and washing (DMF, methanol, and DCM, three times each). To label the peptides with fluorophores, poly (ethylene glycol) (PEG), or biotin, the corresponding dye-COOH, Fmoc-PEG-OH, and biotin were coupled at the N-terminal using the SPPS procedure described above.

For constructing cyclic peptides, Fmoc-propargylglycine-OH (Pra) and Fmoc-azidolysine-OH (Az4) were inserted at the N and C terminals, respectively. A Cu-catalyzed click reaction (CuAAC reaction) was used for cyclization. Specifically, resins were incubated in 20% piperidine/DMF with CuI (2.5 equiv) and L-ascorbic acid (5 equiv) at room temperature overnight. After cyclization, the beads were washed with sodium diethyldithiocarbamate (5% w/v) and DIEA (5% v/v) in DMF to remove the copper catalyst.

To cleave peptides off from the resin, a cleavage solution composed of TFA/TIPS/ddH2O (95:2.5:2.5) was used. The crude peptides were purified by preparative RP-HPLC, and the product purity and identity were confirmed by analytical RP-HPLC and mass spectrometry. RB-cy(YYTYT), C₇₉H₉₇N₁₄O₁₅+, [M+H]⁺ calculated 1481.73, found 1481.62. RB-cy(TYYTY), C₇₉H₉₇N₁₄O₁₅⁺, [M+H]⁺ calculated 1481.73, found 1481.62. RB-cy(YTYYT), C₇₉H₉₇N₁₄O₁₅⁺, [M+H]⁺ calculated 1481.73, found 1481.74. RB-cy(YYYYY), C₈₉H₁₀₁N₁₄O₁₅⁺, [M+H]⁺ calculated 1605.76, found 1605.65. RB-cy(TTTTT), C₆₄H₉₁N₁₄O₁₅⁺, [M+H]⁺ calculated 1295.68, found 1295.62. RB-cy(YSYYS), C₇₇H₉₃N₁₄O₁₅⁺, [M+H]⁺ calculated 1453.69, found 1453.61. RB-cy(SYYSY), C₇₇H₉₃N₁₄O₁₅⁺, [M+H]⁺ calculated 1453.69, found 145367. RB-cy(TAT), C₉₇H1₅₇N₃₆O₁₆⁺ , [M+H]⁺ calculated 2082.26, found 2083.10. AF555-cy(YSYYS), C₇₁H₇₆N₁₄O₂₃S₂²⁻, [M +H]⁺ calculated 1556.47, found 1556.34. AF647-cy(YSYYS), C₈₉H₁₁₃N₁₄O₂₆S₄³⁻, [M+H]⁺ calculated 1921.68, found 1921.67. Fluo-cy(YSYYS), C₆₉H₇₂N₁₂O₁₉, [M+H]⁺ calculated 1372.50, found 1372.25.

Cell lines and cell culture. The human glioblastoma cell line (U87) was gifted from Prof. Wei Wei (Institute For Systems Biology, Seattle). The human monocytic leukemia cell line (THP-1) and the human T-cell leukemia cell line (Jurkat) were purchased from ATCC. The human embryonic kidney cell line (HEK-293T), the human osteosarcoma cell line (U2OS), the human breast cancer cell line (MCF-7), the human metastatic melanoma cell lines (IGR-37, IGR-39, WM266-4 and WM115), the human cervical carcinoma cell line (HeLa), and the human myelogenous leukemia cell line (K562) were gifted from Prof. Yinsheng Wang (UC Riverside). The non-tumorigenic epithelial cell line (MCF 10A) and the human breast adenocarcinoma cell line (MDA-MB-231) were gifted from Prof. Wenwan Zhong (UC Riverside). The canine epithelial kidney cell line (MDCK) and the African green monkey kidney cell line (Vero) were gifted from Prof. Hai Rong (UC Riverside). The human colon carcinoma cell line (HCT116) was gifted from Prof. Xuan Liu (UC Riverside). The human liver carcinoma cell line (HEPG2), the rat liver cell line (MCA7777) and the mouse sarcoma cell line (J774A.1) were gifted from Prof. Joseph Genereux (UC Riverside).

For adherent cell lines, cells were cultured in Dulbecco's modified Eagle's medium (DMEM, Corning) supplemented with 10% heat-inactivated fetal bovine serum (FBS, Gibco) and 100 U/mL penicillin/streptomycin (Sigma). Cells were cultured under 5% CO₂ in a 37° C. incubator. A trypsin-EDTA solution (0.05%, Sigma) was used for passaging once the cells reached 80-90% confluency.

For suspension cell lines, cells were cultured in RPMI 1640, 1× (Corning) medium supplemented with 10% heat-inactivated fetal bovine serum (FBS, Gibco) and 100 U/mL penicillin/streptomycin (Sigma). Cells were cultured under 5% CO₂ in a 37° C. incubator. Fresh culture media was used to dilute old media at a 1:5 ratio after two doubling cycles.

Confocal Imaging experiments. To image the cells, a Zeiss 880 inverted confocal laser scanning microscope (Carl Zeiss, Germany) was used. Image acquisition and analyses were carried out using the manufacturer's software (ZEN, Carl Zeiss). Quantification of fluorescence intensity in single cells was performed using Fiji software.

Flow cytometry experiments. To measure the RB signal intensity, a NovoCyte flow cytometer (NovoExpress) was used. Quantification of fluorescence intensity in each condition was performed using Novoxpress software. Fifty thousand cells were analyzed for each condition. A green, fluorescent dye used in DNA staining, YOYO was included in all the flow cytometry experiments, serving as a cell live/dead indicator. For the YOYO only group, the cells were treated with trypsin for 5-10 min. After that, cells were fixed in a 1.5 ml centrifuge tube using 4% paraformaldehyde at room temperature for 15 min. Subsequently, fixed cells were washed with PBS once and was permeabilized using 90% cold MeOH on ice for 15 min. Then, cells were incubated with YOYO in phenol-free medium. After half an hour, cells were centrifuged to get rid of YOYO solution and resuspended in fresh phenol-free medium before doing flow cytometry.

Dye-EPP incubation. For general incubation, 300 k U87 cells were seeded in full medium containing 10% FBS and 1% PS in a 35×10 mm petri dish. The cells were incubated overnight in full medium. Stock solution of Dye-EPPs were made in DMSO to reach a concentration of 500 μM. Fresh cell culture medium was used to dilute the stock solution to 500 nM and replace the old medium on day 2. Cells were incubated with RB-EPPs for different time periods, followed by a one-time wash with phenol-free fresh culture medium before measurements.

Concentration gradient experiment. U87 Cells were incubated with RB-EPP 6 at different concentrations (20 nM, 200 nM, 2 μM, 20 μM, 50 μM and 100 μM). After wash with fresh medium once cells were examined by confocal imaging.

Time series experiment. U87 Cells were incubated with RB-EPP 6 at 500 nM for 1 h. After wash with fresh medium once, the cells were kept in the 5% CO₂ incubator at 37° C. for different time durations (1 h, 2 h, 6 h and 24 h) before confocal imaging.

Temperature control comparison experiment. U87 cells were kept in the cold room for half an hour before incubated with RB-EPP 6. After that, cells were incubated with RB-EPP 6 at 500 nM at 4° C. Cells in the control group were incubated at 37° C. After 1 h, cells were wash with fresh medium once respectively before confocal imaging.

Endocytosis inhibitors treatment. U87 Cells were incubated with different endocytosis inhibitors for 1 h respectively. Afterwards, old medium was replaced with medium containing 500 nM RB-EPP 6. After 1 h, a trypsin-EDTA solution (0.05%, Sigma) was added to detach the cells. The cells were collected by centrifugation. Fresh phenol-free medium was added to each condition. Once resuspended, cells were filtered into glass tubes before flow cytometry.

Organelle tracker colocalization experiment. Stock solution of trackers for mitochondria, lysosome and ER were diluted with cell culture medium into 100 nM, 500 nM and 4 μM respectively. RB-EPP 6 was added into each working solution at 500 nM. U87 cells were incubated with the both trackers and RB-EPP 6 for 1 h and then washed once before confocal imaging.

Heparin binding assay. U87 cells were treated with Heparin sulfate at 10, 20, 50, 100, 200 μg/ml in 5% CO₂ incubator at 37° C. for 30 min. Afterwards, cell medium was replaced by medium with both heparin sulfate and 500 nM RB-EPP6 for another 1 h incubation. A trypsin-EDTA solution (0.05%, Sigma) was added to detach the cells. The cells were collected by centrifugation. Fresh phenol-free medium was added to each condition. Once resuspended, cells were filtered into glass tubes before flow cytometry.

Octanol-water partition assay. For each RB-EPP, pre-mix 250 μl of octanol with 250 μl of water in a 1.5 ml centrifuge tube. 2 μl of RB-EPP was added to the solution at the concentration of 500 μM. After vortex the mixture, centrifuge at the highest speed for 10 min to separate the octanol and water phases. Samples were collected from both phases and quantified by the Analytical HPLC separately.

RNA-seq studies. In a 100×15 mm dish, 1 m U87 cells were seeded for overnight culture in full DMEM medium containing 10% FBS and 1% PS. RB-EPP 6 diluted with fresh culture medium at 500 nM was used to replace the old medium the next day. After 1 h incubation in the 5% CO₂ incubator at 37° C., the cells were treated with trypsin and resuspended in fresh phenol-free medium after centrifuge. Then, cells were filtered into glass tubes for fluorescence-activated cell sorting (FACS). Cells with the highest and lowest 30% fluorescence intensity was collected separately. RNA extraction was performed using the RNeasy Micro Kit (50) right after FACS experiment.

FIBCD1 competition assay. U87 cells were incubated with 500 nM RB-EPP 6 and YOYO, together with FIBCD1 ligands, sodium acetate (SA) at 3.1 mM (1×) and acetylmannosamine (AMA) at 1.6 mM (1×) respectively. After 1 h, trypsin was added to detach the cells. Cell pallets were collected by centrifugation. Fresh phenol-free medium was added to each condition. Once resuspended, cells were filtered into glass tubes before flow cytometry.

Plasmid transfection assays. 300 k U87 cells were seeded in the 35×10 mm dish for overnight culture. Plasmids (caveolin, 75 ng; clathrin, 100 ng; rab5, 100 ng; rab7a, 10 ng; caveolin-1 CRISPR/Cas9 KO, 3 μg; FIBCD1 CRISPR/Cas9 KO, 3 ug; Control CRISPR, 3 ug) were diluted with Plasmid Transfection Medium (sc-108062) to 50 μl. UltraCruz Transfection Reagent (Santa Cruz Biotechnology) was also diluted with Plasmid Transfection Medium to 50 μl. Plasmids and transfection reagent were mixed well and kept at room temperature for 20 min. Replace the cells with PS free medium and add the mixture to the cells, culture for another 24 h.

MDCK transwell assay. 200 k MDCK cells were seeded in 200 μl in complete growth media to the apical side of a transwell insert (12-well 8 μm pore size Transwell-65 mm). In addition, 1 ml complete growth media is added to the basolateral chamber. Six transwell inserts were prepared for a randomized peptide labeled with RB (RB-GSQTH), and six other inserts are prepared for RB-EPP 6. Incubate the plate for 2-3 days at 37° C. with 5% CO₂. The electrical resistance of MDCK transwell inserts was measured using EVOM Epithelial Voltohmmeter to measure the integrity of tight junctions the following two days. On day 3, serum free media was used to wash both the apical (200 μl) and basolateral (1 ml) wells three times. Cells were kept in a 37° C./5% CO₂ cell incubator equilibrating for 15 min.

The assay was carried out in both apical to basolateral and basolateral to apical direction. Both RB-GSQTH and RB-EPP 6 were prepared at 10 μM in serum free media. For the assay with apical to basolateral direction, the apical chamber was replaced with 200 μl testing compounds. For the assay with basolateral to apical direction, the basolateral chamber was replaced with 1 ml testing compounds. Both plates were incubated in the cell culture incubator for 3 h. After that, 80 μl of the samples were collected from each chamber for RB fluorescence intensity reading using a Synergy H1 microplate reader.

PAMPA assay. All the RB-EPPs were diluted in PBS to 100 μM. 700 μl of the testing peptides were added to the apical chamber of a Corning BioCoat Pre-coated PAMPA Plate. 200 μl PBS was added to the basolateral chamber. The plate was kept at 37° C. with 5% CO₂ for 5 h. After that, 80 μl samples were collected from each chamber for RB fluorescence intensity reading using the Synergy H1 microplate reader.

Lipid binding assay. 95% egg PC, 5% nitrobenzoxadiazole (NBD) were added to 1% cholesterol, PE, PG, ganglioside, SM and PA to make liposomes individually. RB-EPP 6 was diluted with HEPES buffer to different concentrations, ranging from 1 nM to 10 μM. For each lipid type, 400 μl liposomes of 50 μg/ml were added to 400 μl of each RB-EPP 6 concentration. The mixture was incubated at room temperature for 15 min. 80 μl of each condition was collected to measure the fluorescence intensity of NBD suing the Synergy H1 microplate reader.

Ethidium bromide (EB) staining. U87 cells were incubated with 2 μg/ml EB with or without the conjugation of EPP6 for an hour, 3 hours and 6 hours respectively. After 1 h and the cells were washed with fresh phenol-free media once before confocal imaging.

Statistical analysis. Pearson's correlation coefficient was used to calculate correlation between RB and tracker channels in the organelle tracker experiment. Graph generation and statistical analyses were performed using Fiji software.

Documents Cited in this Example:

1. Macara, I. G., Transport into and out of the nucleus. Microbiology and molecular biology reviews 2001, 65 (4), 570-594.

2. Ross, J. L.; Ali, M. Y.; Warshaw, D. M., Cargo transport: molecular motors navigate a complex cytoskeleton. Current opinion in cell biology 2008, 20 (1), 41-47.

3. Mukherjee, S.; Ghosh, R. N.; Maxfield, F. R., Endocytosis. Physiological reviews 1997.

4. Besterman, J. M.; Low, R. B., Endocytosis: a review of mechanisms and plasma membrane dynamics. Biochemical Journal 1983, 210 (1), 1-13.

5. Mettlen, M.; Chen, P. -H.; Srinivasan, S.; Danuser, G.; Schmid, S. L., Regulation of clathrin-mediated endocytosis. Annual review of biochemistry 2018, 87, 871-896.

6. Madsen, S. J.; Hirschberg, H., Macrophages as delivery vehicles for anticancer agents. Therapeutic delivery 2019, 10 (3), 189-201.

7. Bazzani, L.; Donnini, S.; Giachetti, A.; Christofori, G.; Ziche, M., PGE2 mediates EGFR internalization and nuclear translocation via caveolin endocytosis promoting its transcriptional activity and proliferation in human NSCLC cells. Oncotarget 2018, 9 (19), 14939.

8. Peer, D.; Karp, J. M.; Hong, S.; Farokhzad, O. C.; Margalit, R.; Langer, R., Nanocarriers as an emerging platform for cancer therapy. Nature nanotechnology 2007, 2 (12), 751-760.

9. Pattni, B. S.; Chupin, V. V.; Torchilin, V. P., New developments in liposomal drug delivery. Chemical reviews 2015, 115 (19), 10938-10966.

10. Anselmo, A. C.; Mitragotri, S., Nanoparticles in the clinic. Bioengineering& translational medicine 2016, 1 (1), 10-29.

11. Silva, S.; Almeida, A. J.; Vale, N., Combination of cell-penetrating peptides with nanoparticles for therapeutic application: a review. Biomolecules 2019, 9 (1), 22.

12. Gessner, I.; Neundorf, I., Nanoparticles modified with cell-penetrating peptides: Conjugation mechanisms, physicochemical properties, and application in cancer diagnosis and therapy. International journal of molecular sciences 2020, 21 (7), 2536.

13. Zhao, R.; Diop-Bove, N.; Visentin, M.; Goldman, I. D., Mechanisms of membrane transport of folates into cells and across epithelia. Annual review of nutrition 2011, 31, 177-201.

14. Sharma, G.; Lakkadwala, S.; Modgil, A.; Singh, J., The role of cell-penetrating peptide and transferrin on enhanced delivery of drug to brain. International journal of molecular sciences 2016, 17 (6), 806.

15. Smith, B. A.; Daniels, D. S.; Coplin, A. E.; Jordan, G. E.; McGregor, L. M.; Schepartz, A., Minimally cationic cell-permeable miniature proteins via a-helical arginine display. Journal of the American Chemical Society 2008, 130 (10), 2948-2949.

16. Snyder, E. L.; Dowdy, S. F., Cell penetrating peptides in drug delivery. Pharmaceutical research 2004, 21 (3), 389-393.

17. Derakhshankhah, H.; Jafari, S., Cell penetrating peptides: A concise review with emphasis on biomedical applications. Biomedicine& Pharmacotherapy 2018, 108, 1090-1096.

18. Ramsey, J. D.; Flynn, N. H., Cell-penetrating peptides transport therapeutics into cells. Pharmacology& therapeutics 2015, 154, 78-86.

19. Zou, L.; Peng, Q.; Wang, P.; Zhou, B., Progress in research and application of HIV-1 TAT-derived cell-penetrating peptide. Journal of Membrane Biology 2017, 250 (2), 115.

20. Mokhtarieh, A. A.; Kim, S.; Lee, Y.; Chung, B. H.; Lee, M. K., Novel cell penetrating peptides with multiple motifs composed of RGD and its analogs. Biochemical and biophysical research communications 2013, 432 (2), 359-364.

21. Letoha, T.; Gaal, S.; Somlai, C.; Czajlik, A.; Perczel, A.; Penke, B., Membrane translocation of penetratin and its derivatives in different cell lines. Journal of Molecular Recognition 2003, 16 (5), 272-279.

22. Hu, C.; Chen, X.; Huang, Y.; Chen, Y., Co-administration of iRGD with peptide HPRP-A1 to improve anticancer activity and membrane penetrability. Scientific reports 2018, 8 (1), 1-14.

23. Song, J.; Qian, Z.; Sahni, A.; Chen, K.; Pei, D., Cyclic cell-penetrating peptides with single hydrophobic groups. Chembiochem: a European journal of chemical biology 2019, 20 (16), 2085.

24. Habault, J.; Poyet, J. -L., Recent advances in cell penetrating peptide-based anticancer therapies. Molecules 2019, 24 (5), 927.

25. Kang, Z.; Ding, G.; Meng, Z.; Meng, Q., The rational design of cell-penetrating peptides for application in delivery systems. Peptides 2019, 121, 170149.

26. Kurrikoff, K.; Langel, Ü., Recent CPP-based applications in medicine. Expert opinion on drug delivery 2019, 16 (11), 1183-1191.

27. Ye, J.; Liu, E.; Yu, Z.; Pei, X.; Chen, S.; Zhang, P.; Shin, M. -C.; Gong, J.; He, H.; Yang, V. C., CPP-assisted intracellular drug delivery, what is next? International journal of molecular sciences 2016, 17 (11), 1892.

28. Ding, Y.; Cui, W.; Sun, D.; Wang, G. -L.; Hei, Y.; Meng, S.; Chen, J. -H.; Xie, Y.; Wang, Z. -Q., In vivo study of doxorubicin-loaded cell-penetrating peptide-modified pH-sensitive liposomes: biocompatibility, bio-distribution, and pharmacodynamics in BALB/c nude mice bearing human breast tumors. Drug design, development and therapy 2017, 11, 3105.

29. Jones, S. W.; Christison, R.; Bundell, K.; Voyce, C. J.; Brockbank, S. M.; Newham, P.; Lindsay, M. A., Characterisation of cell-penetrating peptide-mediated peptide delivery. British journal of pharmacology 2005, 145 (8), 1093-1102.

30. Qian, Z.; Martyna, A.; Hard, R. L.; Wang, J.; Appiah-Kubi, G.; Coss, C.; Phelps, M. A.; Rossman, J. S.; Pei, D., Discovery and mechanism of highly efficient cyclic cell-penetrating peptides. Biochemistry 2016, 55 (18), 2601-2612.

31. Kalafatovic, D.; Giralt, E., Cell-penetrating peptides: Design strategies beyond primary structure and amphipathicity. Molecules 2017, 22 (11), 1929.

32. Ruseska, I.; Zimmer, A., Internalization mechanisms of cell-penetrating peptides. Beilstein journal of nanotechnology 2020, 11 (1), 101-123.

33. Xu, P.; Van Kirk, E. A.; Zhan, Y.; Murdoch, W. J.; Radosz, M.; Shen, Y., Targeted charge-reversal nanoparticles for nuclear drug delivery. Angewandte Chemie International Edition 2007, 46 (26), 4999-5002.

### Example 2. Exemplary Conjugates

### EPP6-GEM

One exemplary conjugate described herein is made through the conjugation of EPP6 to the chemotherapeutic Gemcitabine. Gemcitabine is a hydrophilic chemotherapeutic that is used to treat a variety of cancers, such as bladder cancer and non-small cell lung cancer (NSCLC). Typically, Gemcitabine is only administered through IV injections and isn't very cell-permeable, so higher dosages are often needed to see a reasonable effect on solid tumors. Due to its potency and off-target toxicity, many uncomfortable side effects come along with its usage.

EPP6-GEM was synthesized using a combination of different synthesis methods; the Fmoc-based solid phase peptide synthesis was adopted for the EPP6¹ while the gemcitabine was prepared using methods from other sources^{2,3}. Gemcitabine HCl and Na₂CO₃ were added to a round bottom flask, at a ratio of 1:5 Gemcitabine to Na₂CO₃ and dissolved in 4 ml of Dioxane and 1 mL of water and stirred using a stir bar. DBDC was added to the reaction mixture, in equimolar amount to Gemcitabine HCl, and stirred at room temp for 48 hours. After the reaction, the resulting solution then had 2 mL of water added and was extracted 2 times using 30 mL of Ethyl Acetate. The organic extracts were washed with 5 mL of water then 5 mL of brine, dried over Na₂SO₄ and concentrated to dryness using Rotary Evaporation. The residue was then subjected to flash chromatography (DCM-acetone-EtOH 1:1:0.02) to give what has been dubbed GEM-BOC MW: 363. After flash chromatography, the sample was confirmed to be synthesized through NMR and ESI-MS. GEM-BOC was then dissolved in 8 ml of dioxane and 10× molar excess of DBDC was added to the reaction mixture. This mixture was then stirred at 37° C. for 70 hours at an rpm of 250. After 70 hours, the solvent was removed using Rotary Evaporation and washed with 2 mL of water. The resulting solids were dried and dissolved, then subjected to flash chromatography (DCM-acetone 9:1 to 4:1) to give GEM-Double BOC MW: 463 (see Scheme 1).

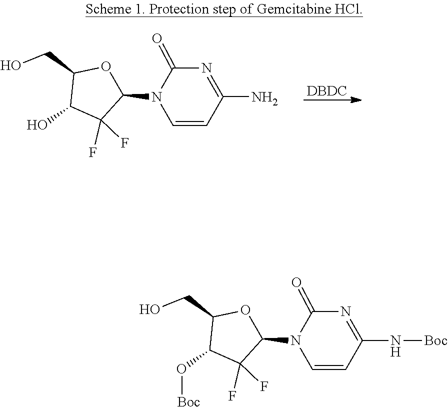 [SMILES: Nc1ccn([C@@H]2O[C@H](CO)[C@@H](O)C2(F)F)c(=O)n1 ; "DBDC" ; CC(C)(C)OC(=O)Nc1ccn([C@@H]2O[C@H](CO)[C@@H](OC(=O)OC(C)(C)C)C2(F)F)c(=O)n1]

After chromatography, the residue was confirmed through ESI-MS and NMR. GEM-Double BOC was then dissolved in DCM, and an 8 times mole ratio of succinic anhydride was added along with a 10 times mole ratio of DIEA to the solution and stirred at room temp overnight (see Scheme 2). The mixture was treated with 10 mL of water, dried through the use of Rotary Evaporation and purified with HPLC. The product was confirmed through both ESI-MS and NMR.

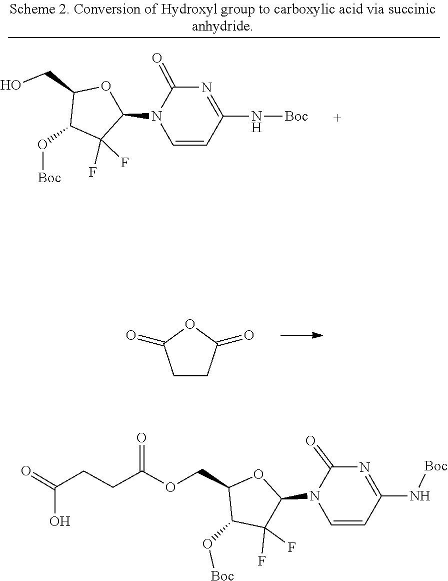 [SMILES: CC(C)(C)OC(=O)Nc1ccn([C@@H]2O[C@H](CO)[C@@H](OC(=O)OC(C)(C)C)C2(F)F)c(=O)n1 ; O=C1CCC(=O)O1 ; CC(C)(C)OC(=O)Nc1ccn([C@@H]2O[C@H](COC(=O)CCC(=O)O)[C@@H](OC(=O)OC(C)(C)C)C2(F)F)c(=O)n1]

EPP6 was synthesized using solid phase Fmoc synthesis and was modulated with an alloc-Lysine (Lys) at the N terminus. This peptide, while still on the bead, was deallylated to remove the alloc protecting group from the Lys and then reacted with protected Gemcitabine using the typical amide bond formation reaction used during peptide synthesis (see Scheme 3). Once confirmed on MALDI the peptide underwent further conjugation with a Rhodamine B Dye and then was cleaved from the bead and purified via HPLC.

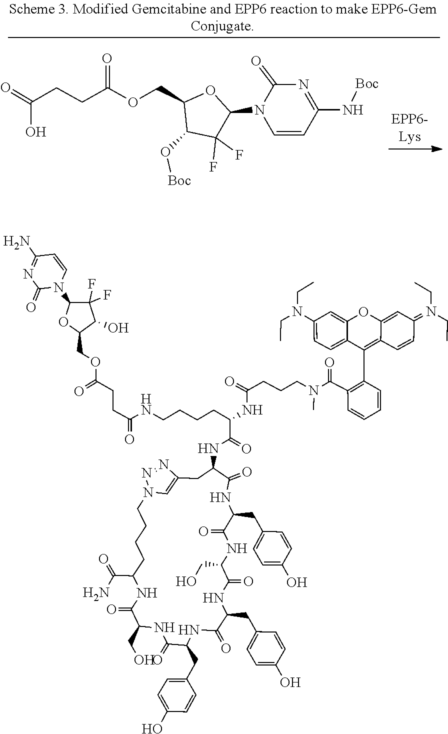

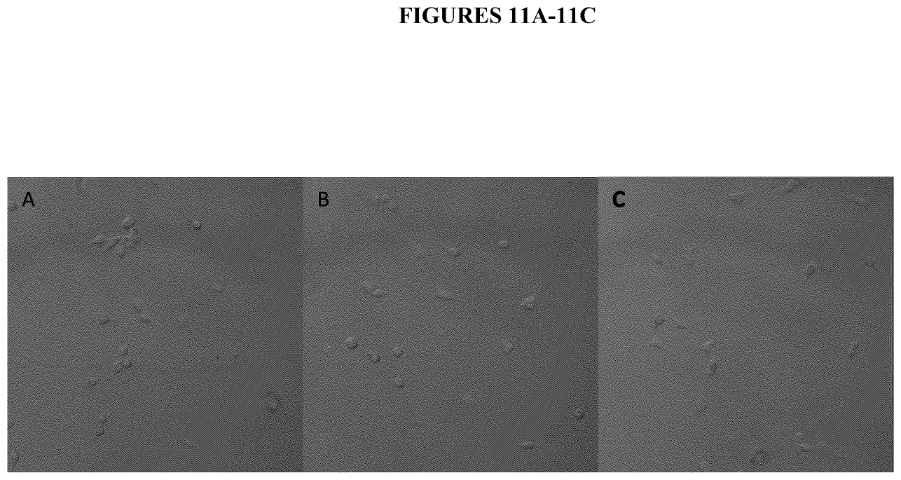

After purification of this conjugate, EPP6-GEM was dissolved in DMSO and diluted to 500 nM in full media (DMEM) and administered to the immortalized glioblastoma cell line U87. These cells were monitored using Confocal Microscopy for internalization of the conjugate. FIGS. 11A-11C show three images of U87 cells with different combinations of dyes internalized. FIG. 11A shows U87 cells with signal from the Hoechst dye, a nucleus staining dye used to outline the cell. FIG. 11B shows the cells with signal from the EPP6-RB and Hoechst dye to serve as a positive control which is the dye conjugated to EPP6 peptide. FIG. 11C shows the signal of both Hoechst and EPP6-GEM. EPP6-GEM has been outfitted with a Rhodamine B dye. Though the positive control of the EPP6 unconjugated to drug is showing much higher signals, the fact that some internalization is occurring from the EPP6-GEM conjugate indicates that the conjugation of the drug to the peptide is not impeding the internalization of the peptide. Cell viability experiments were carried out to determine if the conjugate can kill cells at a similar rate to the free drug or at an improved rate.

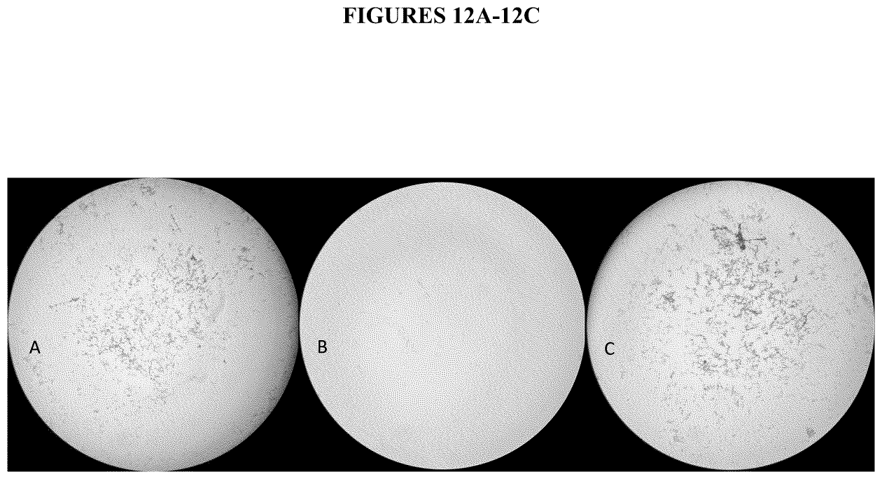

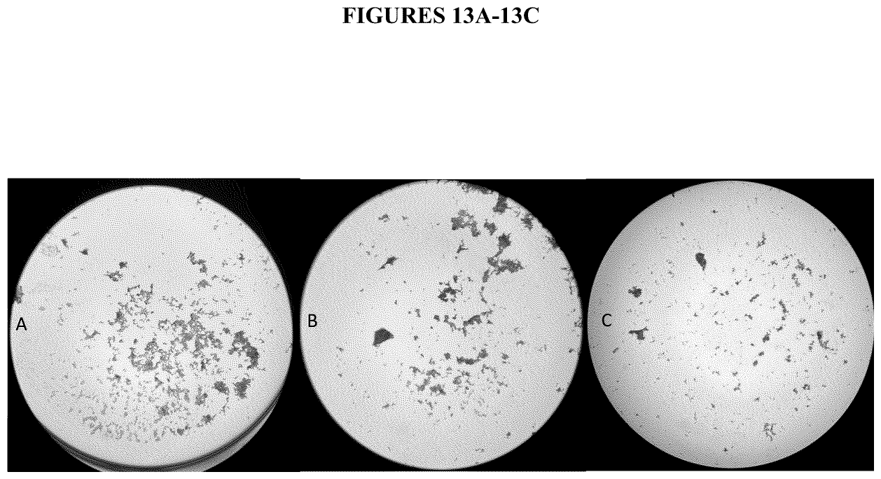

Crystal violet staining is a cell viability test used to determine if a drug or molecule is killing cells based on the difference in cell number from the untreated control. U87 cells are an adherent cell line if they are alive and healthy, they attach securely to the bottom of the petri dish. Herein we passaged U87 cells into a 96-well plate and made quadruplets for each concentration of the drug we planned to use. After staining the cells that remained after treatment with both the drug and the EPP6-GEM conjugate, the cells were then photographed with a cell phone and compared to the control wells (FIGS. 12A-13C).

As shown in FIGS. 12A-12C, the conjugate EPP6-GEM can kill the cells more effectively than the free drug at a biologically relevant concentration. This indicates that the drug is more effective due to its conjugation to the EPP and therefore increased cell permeability. To validate this finding, we wanted to investigate that the increased uptake and cell death were purely due to the EPP6 internalizing and not the more gemcitabine entering the cell of its own accord. So, crystal violet staining was carried out this time using a cell line of U87 that had the FIBCD1 protein removed using the CRISPR-Cas9 knockout procedure (FIGS. 13A-13C). Since the EPP6 can enter the cell through endocytosis via binding to the FIBCD1 membrane protein if the FIBCD1 receptor is removed there should not be the same increased killing effect that we saw previously in FIGS. 12A-12C.

FIGS. 13A-13C show that at the same biologically relevant concentration, 500 nM, the EPP6-GEM conjugate cannot perform significantly better than the free drug in the FIBCD1-KO U87 cell line. This was to be expected as the EPP6 likely enters the cell via endocytosis by binding to the FIBCD1 protein. Since the protein was removed from the cells the EPP6 was not able to enter the cells as effectively, so the cells didn't die at the same rate as they did in the previous experiment (FIGS. 12A-12C). From these two crystal violet staining experiments, there is great promise that the EPP6-GEM conjugate can be an effective and specific drug that can be made into a pill form (e.g., allowing for more controlled dosages to be administered and/or limiting many off-target effects).

### EPP 6-Insulin

The next exemplary conjugate is the conjugation of EPP6 to Human Recombinant Insulin. Insulin is a widely used substance that helps millions of people living with Diabetes. However, Insulin can only be administered through a needle injection in the abdomen and for many patients this must be done quite often. Over time insulin as well as needles and syringes can be quite expensive and uncomfortable to use. By conjugating the EPP6 to insulin we can potentially make this drug into a pill form. Herein we discuss the methods used to validate that this conjugate was synthesized and conjugated to EPP6. ⁴

Four mg of Human Recombinant Insulin were dissolved using a solution of PBS 1× that is in acid pH range of 4-5. This will help solubilize the insulin as it has difficulty solubilizing in basic pH. Then Glycerol, and 100 mM of NaHCO₃ were added to the solution of insulin and the pH was adjusted until it is approximately 8. The ratio between these solvents should be 1:1:1 and the total volume is 450 μL. 12.5 mg of SPDP was dissolved in 260 μL of DMF to make a 50 mg/mL solution of succinimidyl 3-(2-pyridyldithio)propionate (SPDP). The SPDP solution was added to the solution of insulin in 5 μL intervals every 20 minutes for 2 hours. After 2 hours a small amount of the insulin solution was taken and diluted with MeOH before checking MALDI. The mass of Insulin modulated with SPDP should be 6004 g/mol. If the mass signal is not high enough continue to add SPDP until it is. After the reaction the solution was filtered and injected in HPLC for separation using 0-15 min 15-50% ACN & 0.01% TFA, 15-35 min 50-75% ACN & 0.01% TFA, and 35-40 min 75-100% ACN & 0.01% TFA. Once the HPLC run is complete the fractions in MALDI were checked for the appropriate mass then Rotary Evaporation was used to reduce the samples, followed by freezing the sample for lyophilization. Once dried, the sample was dissolved in 1.2 mL of PBS, Glycerol and NaHCO₃ solution, in the same ratios and concentrations as laid out above and equimolar of EPP6 with Cysteine was added in and allow to react for 1-2 hours. After the reaction MALDI was checked to ensure the mass is correct (6928 g/mol) and the reaction product was purified in HPLC using conditions laid out previously and the reduced fraction was lyophilized and collected.

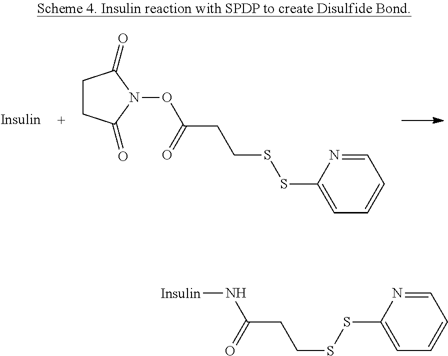

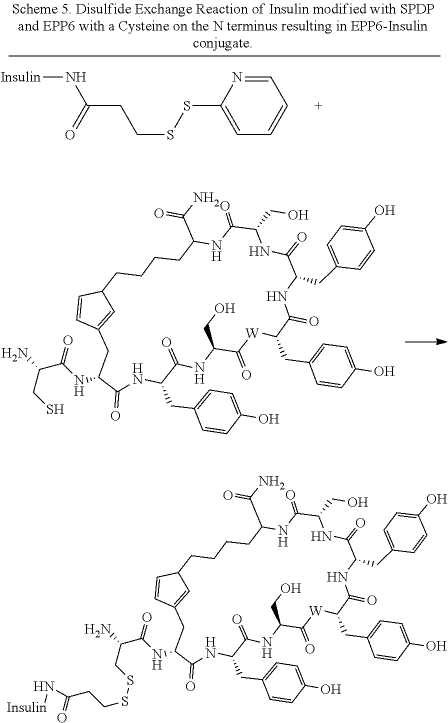

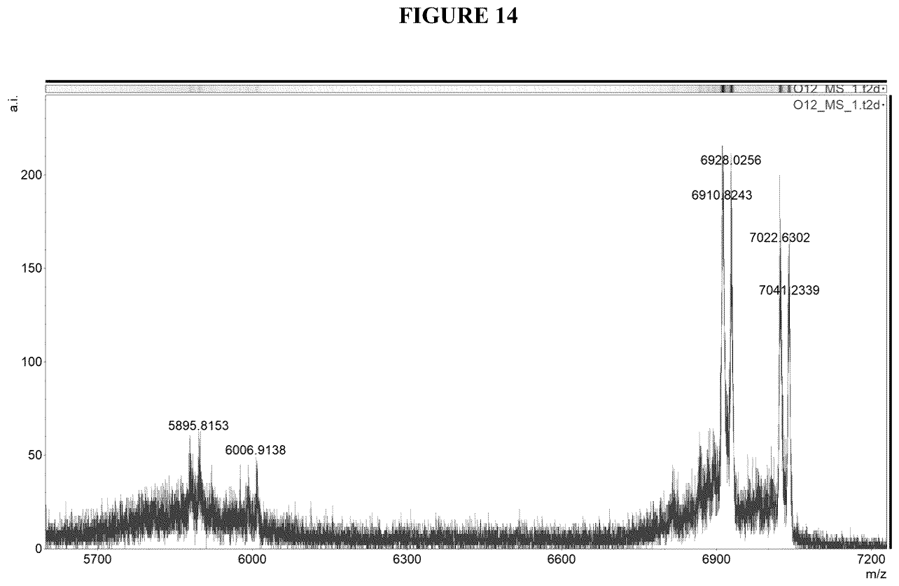

After running these reactions and checking the MALDI we were able to generate the desired product of EPP6 conjugated to insulin (FIG. 14). The reaction is scalable, and a reasonable amount of product can be produced.

From all the data presented herein it is reasonable to conclude that this exemplary peptide, EPP6, can have a significant impact in pharmaceutical and medicinal chemistry research as a means of delivering biologically relevant cargo inside various cell types. EPP6 can form conjugates with drugs such as Gemcitabine and proteins such as Human Recombinant Insulin. It has also been shown herein that the conjugation of this peptide can provide improved effects for the biomolecule attached. These data suggest that this exemplary peptide holds great promise to be used in a clinical setting and potentially improve the delivery of many drugs and proteins to many patients that are in desperate need.

Documents referenced in Example 2:
1. Wang et al. J. Am. Chem. Soc. 2022, 144, 44, 20288-20297
2. Guo et al. J. Org. Chem., Vol. 64, No. 22, 1999
3. Tamvakopoulos et al. Bioconjugate Chem. 2014, 25, 4, 813-823
4. Shen et al. Mol. Pharmaceutics 2009, 6, 2, 492-503

All publications, patents and patent applications cited herein are incorporated herein by reference in their entirety, including Donghyeok Gang, et al, Genes. 2018 Nov.; 9(11): 557; YH Lau, at al, Chem Soc Rev. 2015 Jan. 7;44(1):91-1021; and Siwen Wang et al., J. Am. Chem. Soc. 2022, 144, 20288-20297. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and “or” and similar referents in the context of describing the invention are to be construed to cover both the singular and the plural, unless otherwise indicated herein or clearly contradicted by context.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Variations of those embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention. Further, all publications mentioned herein are incorporated by reference in their entireties.

What is claimed is:

1. A macrocyclic compound comprising a pentapeptide segment X₁-X₂-X₃-X₄-X₅, wherein:
X₁, X₂, X₃, X₄, and X₅ are each independently a residue of Phe, Tyr, Thr, Ser, or homo-Ser;
wherein Phe or Tyr is optionally substituted with one or more halo or hydroxy group on the phenyl ring; and
wherein Thr, Ser, or homo-Ser is optionally substituted with one or more halo, hydroxy, (C₁-C₆)alkyl, (C₃-C₆)cycloalkyl, aryl, or heteroaryl, wherein the (C₁-C₆)alkyl, (C₃-C₆)cycloalkyl, aryl, or heteroaryl is optionally substituted with one or more halo or hydroxy group;
or a peptidyl residue or a salt thereof,
provided the pentapeptide segment is not Tyr-Tyr-Thr-Tyr-Thr (SEQ ID NO:1).

2. The compound of claim 1, wherein X₁ is a residue of Phe or Tyr.

3. The compound of claim 1, wherein X₂ is a residue of Thr, Ser, or homo-Ser.

4. The compound of claim 1, wherein X₃ is a residue of Phe or Tyr.

5. The compound of claim 1, wherein X₄ is a residue of Phe or Tyr.

6. The compound of claim 1 wherein X₅ is a residue of Thr, Ser, or homo-Ser.

7. The compound of claim 1, wherein the pentapeptide segment X₁-X₂-X₃-X₄-X₅ is selected from the group consisting of
(SEQ ID NO: 2)
Thr-Tyr-Tyr-Thr-Tyr,
(SEQ ID NO: 3)
Tyr-Thr-Tyr-Tyr-Thr,
(SEQ ID NO: 4)
Tyr-Tyr-Tyr-Tyr-Tyr,
(SEQ ID NO: 6)
Tyr-Ser-Tyr-Tyr-Ser,
and
(SEQ ID NO: 7)
Ser-Tyr-Tyr-Ser-Tyr.

8. The compound of claim 1, from N terminal to C terminal, having formula I:
(Rₙ)₂N-c[X₀-X₁-X₂-X₃-X₄-X₅-X₆]—C(═O)—R_{c} (I)
wherein:
X₀ is a residue of an amino acid,
X₆ is a residue of an amino acid,
each Rₙ is independently H, (C₁-C₆)alkyl, or (C₁-C₆)alkanoyl, and
R_{c} is hydroxy or —N(R_{f})₂, wherein each R_{f} is independently H or (C₁-C₆)alkyl.

9. The compound of claim 8, comprising a cyclic heptapeptide sequence selected from the group consisting of
(SEQ ID NO: 9)
c[Pra-Thr-Tyr-Tyr-Thr-Tyr-Az4],
(SEQ ID NO: 10)
c[Pra-Tyr-Thr-Tyr-Tyr-Thr-Az4],
(SEQ ID NO: 11)
c[Pra-Tyr-Tyr-Tyr-Tyr-Tyr-Az4],
(SEQ ID NO: 13)
c[Pra-Tyr-Ser-Tyr-Tyr-Ser-Az4],
and
(SEQ ID NO: 14)
c[Pra-Ser-Tyr-Tyr-Ser-Tyr-Az4].

10. The compound of claim 1, having formula II: 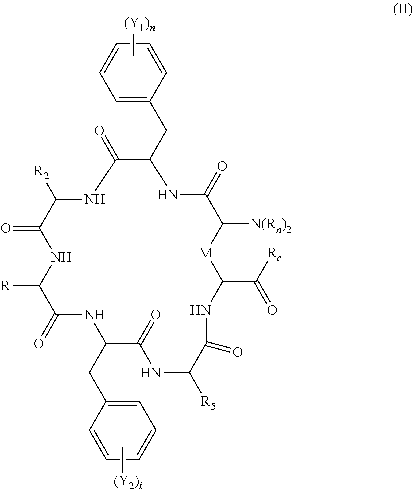
wherein
R₂ and R₅ are each independently (C₁-C₃)alkyl substituted with one or more hydroxy groups,
R is benzyl optionally substituted with one or more halo or hydroxy groups on the phenyl ring, or (C₁-C₃)alkyl substituted with one or more hydroxy groups,
each Y₁ is independently halo or hydroxy,
each Y₂ is independently halo or hydroxy,
h and i are each independently 0, 1, 2, 3, 4, or 5,
each Rₙ is independently H, (C₁-C₆)alkyl, or (C₁-C₆)alkanoyl,
R_{c} is hydroxy or —N(R_{f})₂,
each R_{f} is independently H or (C₁-C₆)alkyl,
M is divalent, branched or unbranched, saturated or unsaturated, hydrocarbon chain having from 2 to 16 carbon atoms, wherein one or more of the carbon atoms is optionally replaced by (—O—), (—S—), (—NRₐ—), (C₃-C₈)cycloalkyl, aryl, or heteroaryl, wherein the chain is optionally substituted on carbon with one or more substituents selected from the group consisting of halo, hydroxy, mercapto, oxo (═)), and
thioxo(═S); wherein Rₐ is a H or (C₁-C₆)alkyl.

11. The compound of claim 10, having formula II(a): 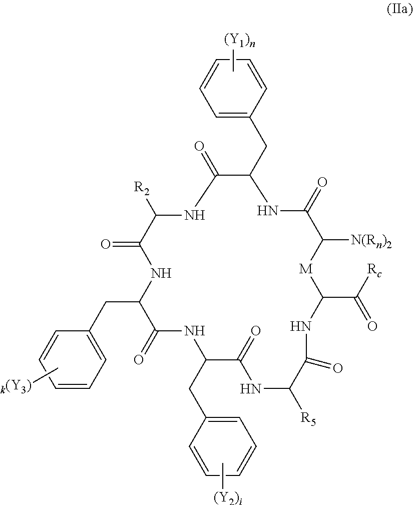
wherein each Y₃ is independently halo or hydroxy, and
k is 0, 1, 2, 3, 4, or 5.

12. The compound of claim 9, comprising a cyclic heptapeptide sequence of
(SEQ ID NO: 10)
c[Pra-Tyr-Thr-Tyr-Tyr-Thr-Az4]
or
(SEQ ID NO: 13)
c[Pra-Tyr-Ser-Tyr-Tyr-Ser-Az4].

13. The compound of claim 10, having the structure of

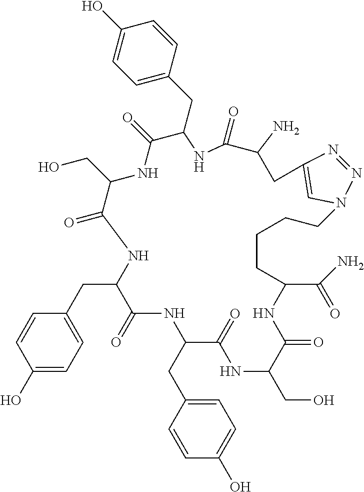 [SMILES: NC(=O)C1CCCCn2cc(nn2)CC(N)C(=O)NC(Cc2ccc(O)cc2)C(=O)NC(CO)C(=O)NC(Cc2ccc(O)cc2)C(=O)NC(Cc2ccc(O)cc2)C(=O)NC(CO)C(=O)N1]

14. A conjugate having the structure of formula (III):
P-L-cargo (III)
wherein:
P is a peptidyl residue of the compound according to claim 1;
cargo is a chemical agent or biological agent (e.g., a detectable agent, synthetic biodegradable polymer, peptide, polypeptide, or polynucleotide);
L is a linking moiety having the structure:
—W—Z—T—
W is selected from the group consisting of a bond, —O—, —S—, —C(═O)—, —N(R_{S})—, —C (═O)NH—, —C(═S)NH—, —C(═O)O—, —C(═O)S—, —NHSO₂—, —OC(═O)NH—, —NHC(═O)NH—, and —NHC(═S)NH—, wherein R_{S} is H or (C₁-C₆)alkyl;
T is selected from the group consisting of a bond, —O—, —S—, —C(═O)—, —N(Rᵤ)—, —C (═O)NH—, —C(═S)NH—, —C(═O)O—, —C(═O)S—, —NHSO₂—, —OC(═O)NH—, —NHC(═O)NH—, and —NHC(═S)NH—, wherein Rᵤ is H or (C₁-C₆)alkyl;
Z is selected from the group consisting of a bond, or a divalent, branched or unbranched, saturated or unsaturated, hydrocarbon chain having from 2 to 16 carbon atoms, wherein one or more of the carbon atoms is optionally replaced by (—O—), (—S—), (—NRₚ—), (C₃-C₈)cycloalkyl, aryl, or heteroaryl, wherein Rₚ is H or (C₁-C₆)alkyl, wherein the hydrocarbon chain is optionally substituted on carbon with one or more substituents selected from the group consisting of halo, hydroxy, amino, mercapto, oxo(═O), and thioxo(═S).

15. The conjugate of claim 14, wherein the cargo is a fluorescent dye, a chemotherapeutic agent (e.g., gemcitabine), or a protein.

16. The conjugate of claim 14, having the structure of 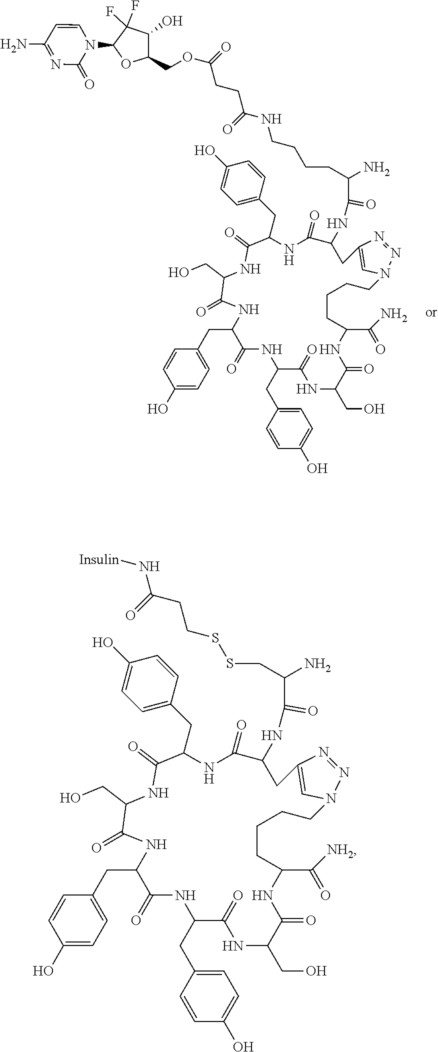
or salt thereof.

17. A conjugate having the structure of formula (IV):
ₘ(P—L [symbol] Cargo (IV)
wherein:
each P is independently a peptidyl residue of the macrocyclic compound according to claim 1;
cargo is a biological agent (e.g., synthetic biodegradable polymer, polypeptide, or polynucleotide);
each L is independently a linking moiety having the structure:
—W—Z—T—
W is selected from the group consisting of a bond, —O—, —S—, —C(═O)—, —C (═O)NH—, —C(═S)NH—, —C(═O)O—, —C(═O)S—, —NHSO₂—, —OC(═)NH—, —NHC(═O)NH—-, and —NHC(═S)NH—, wherein R_{S} is H or (C₁-C₆)alkyl;
T is selected from the group consisting of a bond, —O—, —S—, —C(═O)—, —N(Rᵤ)—, —C (═O)NH—, —C(═S)NH—, —C(═O)O—, —C(═O)S—, —NHSO₂—, —OC(═O)NH—, —NHC(═O)NH—, and —NHC(═S)NH—, wherein Rᵤ is H or (C₁-C₆)alkyl;
Z is selected from the group consisting of a bond, or a divalent, branched or unbranched, saturated or unsaturated, hydrocarbon chain having from 2 to 16 carbon atoms, wherein one or more of the carbon atoms is optionally replaced by (—O—), (—S—), (—NRₚ—), (C₃-C₈)cycloalkyl, aryl, or heteroaryl, wherein Rₚ is H or (C₁-C₆)alkyl, wherein the hydrocarbon chain is optionally substituted on carbon with one or more substituents selected from the group consisting of halo, hydroxy, amino, mercapto, oxo(═O), and thioxo(═S); and
m is an integer that is >=1;
or a salt thereof.

18. A conjugate having the structure of formula (V):
P—L [symbol] Cargo)ₙ (V)
wherein:
P is a peptidyl residue of the macrocyclic compound according to claim 1;
each cargo is independently a chemical or biological agent (e.g., a detectable agent, synthetic biodegradable polymer, peptide, polypeptide, or polynucleotide);
L is a linking moiety having the structure:
—W—Z—T—
W is selected from the group consisting of a bond, —O—, —S—, —C(═O)—, —N(R_{S})—, —C (═O)NH—, —C(═S)NH—, —C(═O)O—, —C(═O)S—, —NHSO₂—, —OC(═O)NH—, —NHC(═O)NH—, and —NHC(═S)NH—, wherein R_{S} is H or (C₁-C₆)alkyl;
T is selected from the group consisting of a bond, —O—, —S—, —C(═O)—, —N(Rᵤ)—, —C (═O)NH—, —C(═S)NH—, —C(═O)—, —C(═O)S—, —NHSO₂—, —OC(═O)NH—, —NHC(═O)NH—, and —NHC(═S)NH—, wherein Rᵤ is H or (C₁-C₆)alkyl;
Z is selected from the group consisting of a bond, or a divalent, branched or unbranched, saturated or unsaturated, hydrocarbon chain having from 2 to 16 carbon atoms, wherein one or more of the carbon atoms is optionally replaced by (—O—), (—S—), (—NRₚ—), (C₃-C₈)cycloalkyl, aryl, or heteroaryl, wherein Rₚ is H or (C₁-C₆)alkyl, wherein the hydrocarbon chain is optionally substituted on carbon with one or more substituents selected from the group consisting of halo, hydroxy, amino, mercapto, oxo(═O), and thioxo(═S); and
n is an integer that is >=1;
or a salt thereof.

19. A method for intracellular delivery, comprising contacting a cell with the compound according to claim 1.

20. A method for treating a disease or condition, comprising administering the conjugate of claim 14 to a subject in need thereof, wherein the cargo is a therapeutic agent.
